SEQUENCE LISTING (21 sequences)

SEQ ID NO: 1 - DNA, 27 bp, Artificial Sequence
  Synthetic polynucleotide
tctgtgtggtggtgcggcgcgcatgcc

SEQ ID NO: 2 - DNA, 36 bp, Artificial Sequence
  Synthetic polynucleotide
atccaggcccagcagtgggtttgggattggtttgcc

SEQ ID NO: 3 - protein, 19 aa, Artificial Sequence
  Synthetic polypeptide
CARYYYDSSGYYADAFDIW

SEQ ID NO: 4 - protein, 11 aa, Artificial Sequence
  Synthetic polypeptide
CAKGIAAVDYW

SEQ ID NO: 5 - protein, 21 aa, Artificial Sequence
  Synthetic polypeptide
CASLRSAYYYDSSGRDAFDIW

SEQ ID NO: 6 - protein, 9 aa, Artificial Sequence
  Synthetic polypeptide
CAKVRGGDW

SEQ ID NO: 7 - protein, 19 aa, Artificial Sequence
  Synthetic polypeptide
CARGGQLSSGYYFDAFDIW

SEQ ID NO: 8 - protein, 13 aa, Artificial Sequence
  Synthetic polypeptide
CARDVGEVPFDLW

SEQ ID NO: 9 - protein, 18 aa, Artificial Sequence
  Synthetic polypeptide
CAHMYYYDSSGYYYFDYW

SEQ ID NO: 10 - protein, 11 aa, Artificial Sequence
  Synthetic polypeptide
CAKGVSGMDVW

SEQ ID NO: 11 - protein, 12 aa, Artificial Sequence
  Synthetic polypeptide
CAKGLGYGMDVW

SEQ ID NO: 12 - protein, 11 aa, Artificial Sequence
  Synthetic polypeptide
CAKGIARFDYW

SEQ ID NO: 13 - protein, 25 aa, Artificial Sequence
  Synthetic polypeptide
CARDGVPETYSCSGGSCWTDAFDIW

SEQ ID NO: 14 - protein, 12 aa, Artificial Sequence
  Synthetic polypeptide
CAKGGFGAFDIW

SEQ ID NO: 15 - protein, 11 aa, Artificial Sequence
  Synthetic polypeptide
CARGVTTPDYW

SEQ ID NO: 16 - protein, 11 aa, Artificial Sequence
  Synthetic polypeptide
CARNANYYGYW

SEQ ID NO: 17 - protein, 21 aa, Artificial Sequence
  Synthetic polypeptide
CASLRSAYYHDSSGRDAFDIW

SEQ ID NO: 18 - protein, 11 aa, Artificial Sequence
  Synthetic polypeptide
CARGGGIAARW

SEQ ID NO: 19 - protein, 11 aa, Artificial Sequence
  Synthetic polypeptide
CARRASAFDIW

SEQ ID NO: 20 - protein, 16 aa, Artificial Sequence
  Synthetic polypeptide
CARGPGGVGATVLDYW

SEQ ID NO: 21 - protein, 9 aa, Artificial Sequence
  Synthetic polypeptide
CARGRMDVW

(19) United States
(12) Patent Application Publication
(10) Pub. No.: US 2013/0085072 A1
(43) Pub. Date: Apr. 4, 2013

(54) RECOMBINANT RENEWABLE POLYCLONAL ANTIBODIES

(71) Applicant: LOS ALAMOS NATIONAL SECURITY, LLC, Los Alamos, CA (US)

(72) Inventors: Andrew M. Bradbury, Santa Fe, NM (US); Csaba Kiss, Los Alamos, NM (US); Sara D'Angelo, Los Alamos, NM (US); Fortunato Ferrara, Los Alamos, NM (US); Leslie A. Naranjo, Los Alamos, NM (US); Tiziano Gaiotto, Los Alamos, NM (US)

(73) Assignee: LOS ALAMOS NATIONAL SECURITY, LLC, Los Alamos, NM (US)

(21) Appl. No.: 13/631,331

(22) Filed: Sep. 28, 2012

Related U.S. Application Data
(60) Provisional application No. 61/560,589, filed on Nov. 16, 2011.
(60) Provisional application No. 61/542,734, filed on Oct. 3, 2011.

(51) Int. Cl. C40B 30/04; C40B 40/10; C07K 16/00; C40B 10/00

(52) U.S. Cl. 506/1; 506/9; 506/18; 530/387.1

(57) ABSTRACT

Described herein are methods that combine phage and yeast display to create polyclonal antibodies that are renewable, and when amplified over 100 million fold, maintain diversity without loss of representation of any of the antibodies present. The antibody representation remains essentially constant, as confirmed by deep sequencing. The provided methods allow generation, use and propagation of polyclonal antibodies, without concern that representation is lost. Furthermore, because the derivation of the polyclonal pool is carried out in vitro using phage and yeast display, it is possible in various embodiments to eliminate reactivities that are considered undesirable. Additionally, the polyclonal pool can be enriched for higher affinity antibodies.

### CROSS REFERENCE TO RELATED APPLICATIONS

This application claims the benefit of U.S. Provisional Application No. 61/560,589, filed Nov. 16, 2011, and U.S. Provisional Application No. 61/542,734, filed Oct. 3, 2011, both of which are herein incorporated by reference in their entirety.

### ACKNOWLEDGMENT OF GOVERNMENT SUPPORT

This invention was made with U.S. government support under Contract No. DE-AC52-06NA25396 awarded by the U.S. Department of Energy. The U.S. government has certain rights in the invention.

### FIELD

This disclosure concerns recombinant polyclonal antibodies that can be renewed and amplified without loss of representation of any of the antibodies present.

### BACKGROUND

The human genome is estimated to contain 20,000-25,000 genes. When alternative splicing and posttranslational modifications are taken into account, these genes likely encode millions of different protein forms. The most common way to study gene products has traditionally been with affinity reagents, of which antibodies are the prototypical class. Antibodies have been used to identify, localize and quantify proteins in complexes, cells and tissues under normal and diseased conditions, using a variety of different techniques. In addition to their use in basic research, antibodies play important roles in diagnostics and more recently in therapeutics. However, for a very large number of these genes, antibodies are lacking. For example, approximately 500 genes encoding kinases have been identified in the human genome (Milanesi et al., BMC Bioinformatics 6(4):520, 2005; Braconi Quintaje and Orchard, Mol Cell Proteomics 7:1409, 2008; Manning et al., Science 298:1912, 2002), and yet one of the most comprehensive commercial antibody sources does not sell antibodies for over 80% of these.

It is perhaps not surprising that publication rates tend to track those kinases for which antibodies are available, and the most popular kinases have the greatest number of commercially available antibodies. However, this does not mean that the approximately 400 kinases for which antibodies are not commercially available have no biological significance. For example, LRRK2 is a kinase now considered to play an important role in the pathogenesis of Parkinson's disease (Paisan-Ruiz et al., Neuron 44:595, 2004; Zimprich et al., Neuron 44:601, 2004), and yet before 2004 it had never been described in a publication, and would have been considered one of those 400 kinases for which there was no commercial value for selling antibodies. There will be many other proteins in the same category, thus there remains a need for access to well-characterized antibodies for a large number of target antigens. The availability of high quality single antibodies against every human protein will allow for proteome wide assessments under a wide variety of diseases and physiological processes. The results will be applicable to diagnostics, the development of therapeutics and therapeutic markers, as well as to studies of normal and pathological physiological processes.

One critical issue is antibody format. Monoclonal antibodies have the great advantage that they are renewable, but the disadvantage that they are expensive to generate and are usually not functional in all assays. On the other hand, polyclonal antibodies tend to work in all assays, but are not renewable when generated using currently available techniques. While antibody responses to numerous antigens can be generated in many animals, there have been many cases when particularly useful polyclonal antibodies, unique to a critical animal are lost upon the animal's death. Another problem with polyclonal antibodies is their greater tendency to show non-specific binding, a drawback that can be dramatically reduced by affinity purification on the immunizing antigen.

Antibody fragments generated using in vitro methods (e.g. phage/yeast display) have a number of significant advantages, including the ability to target precise epitopes or conformational forms since selection is carried out in vitro. In addition, antibody fragments can be archived as sequences and/or plasmids; their affinities and/or specificities can be improved by subsequent in vitro evolution; and they can be fused to additional functional elements, such as enzymes or antibody constant regions.

### SUMMARY

Disclosed herein are methods of producing recombinant polyclonal antibodies that specifically bind a target antigen, and can be renewed and amplified without significant or detrimental loss of representation of any of the antibodies present.

Provided is a method of generating renewable polyclonal antibodies specific for a target antigen. In some embodiments, the method includes providing a collection of antibody clones; selecting a population of antibody clones from the collection that bind the target antigen; transferring the selected population of antibody clones into a yeast display vector to generate a yeast display antibody library; and selecting a population of yeast clones from the yeast display antibody library that specifically bind the target antigen to generate a target-specific polyclonal antibody population. In some examples, the method further includes expressing the target-specific polyclonal antibody population in an expression vector and/or amplifying the target-specific polyclonal antibody population, wherein amplification of the target-specific polyclonal antibody population maintains at least 70% of the original diversity of the individual yeast clones.

In another embodiment, provided herein is a method of generating a renewable polyclonal antibody population specific for a target antigen, wherein the method includes providing a collection of antibody clones in a first display platform; selecting from the collection a population of antibody clones that bind the target antigen to generate a first antibody display library; transferring the selected population of antibody clones into a second display platform to generate a second antibody display library; and selecting from the second antibody display library a population of clones that specifically bind the target antigen to generate a target-specific polyclonal antibody population. In some examples, the first and second display platforms are selected from phage display, yeast display, ribosome display, bacterial display and in vitro display. In one non-limiting example, the first display platform comprises phage display and the second display platform comprises yeast display.

Also provided are recombinant renewal polyclonal antibodies produced according to any of the methods disclosed herein.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Phage and yeast display selection. One, two or three rounds of phage display selection were carried out before cloning the outputs into the yeast display vector. The x-axis indicates antibody display levels by yeast, while the y-axis indicates the amount of antigen binding. Pre-sort shows the pattern of antigen binding and display immediately following cloning. The sort gates are indicated, and post-sort shows the pattern of antigen binding after one round of sorting.

FIG. 2: Maintenance of diversity in polyclonal pools assessed by 454 sequencing. Percentage prevalence of the top 20 VH sequences in polyclonal yeast antibody pools after amplification. 1 yeast: polyclonal pool after one round of phage selection and one round of yeast selection; 2 yeast: polyclonal pool after one round of phage selection and two rounds of yeast selection; numbers (10,000, 1,000,000 and 100,000,000) indicate the fold amplification of the yeast culture from 2 yeast (e.g. 10,000 indicates that 1 ml of the yeast 2 culture was amplified to the equivalent of 10 liters). Each line represents the percentage prevalence of a single VH sequence in each of the different polyclonal pools.

FIG. 3: Proposed antibody selection pipeline. In tier 1 selections, each microtiter well contains a selection (phage or yeast) against one target, with the final output comprising a monospecific polyclonal antibody pool recognizing the target. Tier 2 selections are carried out by: 1) deep sequencing the output of a single tier 1 selection (in one well) identifying the top 20 clones, and using the sequence to design HCDR3 primers to amplify the secretory plasmids by inverse PCR; or 2) picking 96 random clones and analyzing for specific binding by flow cytometry, and uniqueness by sequencing. Tier 3 selections are similar to tier 2 except that diversity is derived from mutation of a tier 2 clone. Shaded wells indicate positive clones, with intensity corresponding to the level of positivity.

FIGS. 4A-4E: Yeast cell analysis before and after sorting. FIG. 4A shows the yeast population after cloning the Ag85 selected phage display output. The upper right quadrant (Q2) representing those yeast that are displaying scFv and binding antigen contains 2.1% of all yeast cells, of which the top 1%, as indicated by the sort gate, were sorted. FIG. 4B shows the outcome of the first sort after yeast were collected and grown up. Q2 now contains 25.1% of all yeast cells, of which the top 1% were again sorted as shown. FIG. 4C shows the outcome of the second sort: Q2 now contains 35.5% of all yeast cells. FIG. 4D and FIG. 4E show the background streptavidin binding clones that represent, for both the first and the second sort, 3.2% of the total population.

FIGS. 5A-5B: Ag85 detection limits in a sandwich assay. 10⁶ yeast displaying the indicated antibodies were induced, washed and resuspended in serial antigen dilutions. Binding was detected by flow cytometry following the addition of the indicated scFv-Fc fusion antibodies as induced culture supernatants, and fluorescent anti-rabbit secondary reagents. The indicated detection limits are defined as the minimal antigen concentration giving a signal three times greater than background. (A) Detection in phosphate buffered saline. (B) Detection in 1:50 human serum diluted in PBS.

FIG. 6: Stability test of yeast expressed scFv-Fc fusions. Unpurified scFv-Fc fusions were tested for binding to Ag85 by ELISA (Ag85 was adsorbed to plastic) at regular periods over 4 weeks. Results are expressed as percent signals obtained on the first day.

FIG. 7: Optimizing phage and yeast selection. One (top panel) or two (bottom panel) rounds of phage selection were carried out against ubiquitin. After selection, outputs were cloned into a yeast display vector and analyzed by flow cytometry. Yeast displaying antibodies recognizing ubiquitin are found in the upper right quadrant (Q2). The enrichment in positive clones is shown after one or two rounds of yeast sorting carried out on the phage population selected twice on ubiquitin.

FIGS. 8A-8D: Assessment of anti-ubiquitin polyclonal antibodies. (A) ELISA signals for ubiquitin polyclonals. (B) Tables describing component monoclonals of a selected anti-ubiquitin polyclonal. The top table shows the number of clusters of different heavy chain CDR3 sequences identified by Ion torrent sequencing, to different percentage coverage. The bottom table provides the percentage abundance and ranking of each clone as determined for each HCDR3. The HCDR3 sequence for each clone is also shown (SEQ ID NOs: 3-21). Affinities were determined for each monoclonal by yeast display. (C) Correlation between rank abundance and affinity for individual monoclonal antibodies displayed on yeast. (D) The rrpAb displayed on yeast, after cloning into a secretion vector, and after each of four amplification rounds of 100-fold were sequenced and clones identified and quantified. The abundance of each individual clone is indicated after cloning and each amplification step.

FIGS. 9A-9C: Specificity analysis of rrpAbs selected against eight different protein targets. (A) Specificity assessed by ELISA. Six of the eight rrpAbs are tested against all eight different antigens. (B) Western blotting specificity. Four of the antibodies were tested against the congruent antigen (left lane) or against a mixture of the remaining seven antigens (right lane). (C) Deep sequencing analysis of selection outputs. HCDR3 diversity after 1 or 2 sorts for each antigen. The total number of sequences is indicated, as well as the total number of clusters (100%) and the number of clusters comprising 80, 90, 95 or 99% total sequences. For CTBP1, the sorting output for CTBP2 (after two sorts) was sorted negatively against CTBP1 (N1 and N2) and then positively against CTBP2 again.

FIGS. 10A-10D: Eliminating cross-reactivity by appropriate flow sorting. (A) rrpAb displayed on yeast, sorted against CTBP2 tested for binding to CTBP2 and CTBP1. (B) rrpAb displayed on yeast sorted positively against CTBP2 and negatively against CTBP1 tested for binding to CTBP2 and CTBP1. (C) rrpAb displayed on yeast sorted positively against CTBP2 and negatively against CTBP1, followed by an additional positive sort against CTBP2, tested for binding to CTBP2 and CTBP1. (D) ELISA signals for the different rrpAbs in FIGS. 10A-10C tested against CTBP1, 2 and other antigens.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Sep. 25, 2012, 5.11 KB, which is incorporated by reference herein. In the accompanying sequence listing:

SEQ ID NOs: 1 and 2 are primer sequences.

SEQ ID NOs: 3-21 are HCDR3 amino acid sequences.

### DETAILED DESCRIPTION

### I. Abbreviations

Ab antibody

Ag85 antigen 85

APC allophycocyanin

CDR complementarity determining region

CSSA cell surface secretion assay

ELISA enzyme linked immunosorbent assay

FACS fluorescence activated cell sorting

GFP green fluorescent protein

HPA Human Protein Atlas

HRP horseradish peroxidase

HT high throughput

IVTT in vitro transcription translation

mAb monoclonal antibody

ORF open reading frame

PAGE polyacrylamide gel electrophoresis

PE phycoerythrin

PrEST Protein Epitope Signature Tag

QC quality control

rrpAb recombinant renewable polyclonal antibody

scFv single chain variable fragment

SGC Structural Genomics Consortium

TB tuberculosis

VLP virus like particle

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Alternative display platform: In the context of the present disclosure, an “alternative display platform” is any antibody display platform except for yeast display. For example, alternative display platforms include, but are not limited to, phage display and ribosome display.

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kDa) and one “heavy” (about 50-70 kDa) chain. The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains. Each light chain contains a single constant domain (CL), while each heavy chain contains three constant domains, CH1, CH2 and CH3 (or four constant domains for IgE and IgM).

As used herein, the term “antibodies” includes intact immunoglobulins as well as a number of well-characterized fragments having a molecular weight of about 25 to 100 kD. For instance, Fabs, Fvs, and single-chain Fvs (scFvs) that bind to target protein (or an epitope within a protein or fusion protein) would also be specific binding agents for that protein (or epitope). These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) scFv, single chain antibody, a genetically engineered molecule containing the variable region of the light chain, the variable region of the heavy chain, linked by a suitable polypeptide linker as a genetically fused single chain molecule. Methods of making these fragments are routine (see, for example, Harlow and Lane, Using Antibodies: A Laboratory Manual, CSHL, New York, 1999).

Antibodies can be monoclonal or polyclonal. Merely by way of example, monoclonal antibodies can be prepared from murine hybridomas according to the classical method of Kohler and Milstein (Nature 256:495-97, 1975) or derivative methods thereof. Detailed procedures for monoclonal antibody production are described, for example, by Harlow and Lane (Using Antibodies: A Laboratory Manual, CSHL, New York, 1999).

Antigen: A compound, composition, or substance that can stimulate the production of antibodies or a T-cell response in an animal, including compositions that are injected or absorbed into an animal. An antigen reacts with the products of specific humoral or cellular immunity.

Binding affinity: The strength of binding between a binding site and a ligand (for example, between an antibody, and an antigen). The affinity of a binding site X for a ligand Y is represented by the dissociation constant (K_{d}), which is the concentration of Y that is required to occupy half of the binding sites of X present in a solution. A lower (K_{d}) indicates a stronger or higher-affinity interaction between X and Y and a lower concentration of ligand is needed to occupy the sites. In general, binding affinity can be affected by the alteration, modification and/or substitution of one or more amino acids in the epitope recognized by the paratope (portion of the molecule that recognizes the epitope). Binding affinity can be the affinity of antibody binding an antigen. In one example, binding affinity is measured by end-point titration in an Ag-ELISA assay. Binding affinity is substantially lowered (or measurably reduced) by the modification and/or substitution of one or more amino acids in the epitope recognized by the antibody paratope if the end-point titer of a specific antibody for the modified/substituted epitope differs by at least 4-fold, such as at least 10-fold, at least 100-fold or greater, as compared to the unaltered epitope.

Contacting: Placement in direct physical association, which includes both in solid and in liquid form.

Isolated: An “isolated” biological component (such as a nucleic acid molecule or protein) has been substantially separated or purified away from other biological components from which the component naturally occurs (for example, other biological components of a cell), such as other chromosomal and extra-chromosomal DNA and RNA and proteins, including other antibodies. Nucleic acids and proteins that have been “isolated” include nucleic acids and proteins purified by standard purification methods. An “isolated antibody” is an antibody that has been substantially separated or purified away from other proteins or biological components such that its antigen specificity is maintained. The term also embraces nucleic acids and proteins prepared by recombinant expression in a host cell, as well as chemically synthesized nucleic acids or proteins, or fragments thereof.

Label: A detectable compound or composition that is conjugated directly or indirectly to another molecule to facilitate detection of that molecule. Specific, non-limiting examples of labels include fluorescent tags, enzymatic linkages, and radioactive isotopes.

Natural antibody library: An antibody library that comprises antibody sequences generated in vivo. For example, a natural antibody library can include variable domain sequences obtained from human lymphocytes.

Recombinant: A recombinant nucleic acid, polypeptide or antibody is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques.

Regulatable promoter: A promoter that can be controlled by the presence or absence of a specific molecule (e.g., an amino acid, protein, carbohydrate, antibiotic etc.). An inducible promoter is a promoter that is activated by the presence of a particular molecule, whereas a repressible promoter is a promoter that is inhibited by the presence of a particular molecule. For example, the MET25 promoter is repressed in the presence of methionine; the GAL1 promoter is repressed by glucose and is activated by galactose.

Renewable polyclonal antibody population: A population of antibodies that can be amplified without significant or detrimental loss of representation of any of the antibodies present. In the context of the present disclosure, a renewable polyclonal antibody population can generally be amplified 10²- to 10⁸-fold and maintain at least 70% of the original diversity of the individual antibody clones. In some examples, at least 75%, at least 80% or at least 85% of the original diversity of the individual antibody clones is maintained following amplification.

Ribosome display: A technique used to perform in vitro protein evolution to create proteins that bind to a specific ligand. The process results in translated proteins that are associated with their mRNA progenitor which is used, as a complex, to bind to an immobilized ligand in a selection step. The mRNA-protein hybrids that bind well are then reverse transcribed to cDNA and their sequence amplified via PCR. The end result is a nucleotide sequence that can be used to create tightly binding proteins.

Phage display: A method for the study of protein-protein, protein-peptide, and protein-DNA interactions that uses bacteriophages to connect proteins with the genetic information that encodes them. Phage display is often performed using filamentous phage, such as M13, Fd and F1. Most commonly, libraries encoding polypeptides to be displayed are inserted into either gIII or gVIII of the phage, forming a fusion protein (see, e.g., PCT Publication Nos. WO 91/19818; WO 91/18989; WO 92/01047; WO 92/06204; and WO 92/18619). Such a fusion protein typically comprises a signal sequence, usually from a secreted protein other than the phage coat protein, a polypeptide to be displayed and either the gene III or gene VIII protein or a fragment thereof effective to display the polypeptide.

Protein A: A bacterial adhesion protein that was first identified in the cell wall of Staphylococcus aureus, which binds the Fc region of an antibody, particularly IgG molecules. Protein A can be used to identify and select correctly folded VH domains.

Protein L: A bacterial adhesion protein that was first isolated from the surface of Peptostreptococcus magnus, which binds to the kappa light chain of immunoglobulin molecules. Protein L can be used to identify and select correctly folded VL domains.

Single-scaffold library: A synthetic antibody library based on a single antibody framework.

Specific for a target antigen: An antibody that is specific for a target antigen or that specifically binds a target antigen is an antibody that primarily binds the target antigen without significant binding to other antigens.

Synthetic antibody library: An antibody library that is produced by introducing randomized sequences in the antibody binding site of particular frameworks.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

Yeast display: A method that can be used for detecting protein-protein interactions and/or selection of high affinity binding molecules (such as antibodies). Yeast display libraries have been described (see, e.g., Boder and Wittrup, Nat Biotechnol 15:553-557, 1997). Yeast surface expression systems can be used to express recombinant proteins on the surface of yeast, such as S. cerevisiae, as a fusion with a yeast protein, such as Aga-2. Yeast expression can provide correct post-translational modification, processing and folding of mammalian proteins, coupled with rapid characterization of binding affinities of interacting proteins. The expressed fusion proteins can also contain a tag, such as c-myc and HA tag sequences, allowing quantification of the library surface expression by flow cytometry.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Introduction and Overview

Standard polyclonal antibodies constitute many different antibodies recognizing a single target. They are not considered renewable, as each time an animal is immunized, it responds slightly differently, and consequently the composition of antibodies is different. In contrast to traditional polyclonal antibodies, described herein are methods that combine phage and yeast display to create polyclonal antibodies that are renewable, when amplified over 100 million fold. The antibody representation remains essentially constant, as confirmed by deep sequencing.

The provided methods allow generation, use and propagation of polyclonal antibodies, without concern that representation is lost. Furthermore, because the derivation of the polyclonal pool is carried out in vitro using phage and yeast display, it is possible in various embodiments to eliminate reactivities that are considered undesirable—e.g., because they are cross-reactive. Additionally, the polyclonal pool can be enriched for higher affinity antibodies.

Without intended to be limited in any way, antibodies (both individual and as polyclonal pools) generated or maintained using methods described herein can be used in any methods in which traditional antibodies are used. For instance, the described antibodies (and polyclonal pools of antibodies) can be used in research, in therapeutic applications, in detection of known molecules, and so forth.

The methods disclosed herein can be used to create recombinant polyclonal antibodies recognizing a target of interest, in which the recombinant polyclonal antibody can be renewed and amplified without significant or detrimental loss of representation of any of the antibodies present. For example, in particular embodiments, the polyclonal antibody population is amplified, such as amplified at least 10²-fold to 10⁸-fold, and maintains at least 70% of the original diversity of the individual antibody clones. Also provided are antibodies, including polyclonal antibody populations, produced by such methods.

Also disclosed herein is a high-throughput, three-tiered antibody selection pipeline. Tier one antibodies are renewable recombinant polyclonal antibodies that can be amplified from 1 ml to ≧100,000 liters with no loss of diversity. In certain embodiments, they are selected against all human proteins. Structurally, the provided antibodies are similar to traditional antibodies, allowing scientists to use them without modifying protocols. However, unlike traditional polyclonals, they resemble affinity-purified polyclonal antibodies, with most antibodies directed towards the target of interest. These tier one polyclonals are likely to be sufficient for most applications, including most research needs.

Optionally, second tier monoclonal antibodies may be isolated from first tier polyclonal antibody pools. Third tier monoclonal antibodies can be created by making derivative libraries from second tier antibody leads, and offer the selection of monoclonals with improved affinities or specificities. In some embodiments, the disclosed methods combine phage and yeast antibody display, with initial selections carried out from phage antibody libraries, and subsequent selections from yeast libraries created by cloning phage outputs into yeast display vectors. Polyclonal pools can be expressed, for example, as single gene scFv-Fc fusions, using murine or rabbit Fc domains evolved for high expression levels. After internal antibody validation, characterization can be carried out at the Human Protein Atlas, which has an extensive pipeline characterizing antibodies for specificity, tissue and cellular distribution. The renewable polyclonals are highly functional, and provide a source for monoclonal antibodies when needed.

### IV. Overview of Several Embodiments

Provided herein are methods of generating renewable polyclonal antibodies specific for a target antigen.

In some embodiments, the method includes providing a collection of antibody clones; selecting a population of antibody clones from the collection that bind the target antigen; transferring the selected population of antibody clones into a yeast display vector to generate a yeast display antibody library; and selecting a population of yeast clones from the yeast display antibody library that specifically bind the target antigen to generate a target-specific polyclonal antibody population.

In some embodiments, the method of generating renewable polyclonal antibodies specific for a target antigen further includes expressing the target-specific polyclonal antibody population in an expression vector. In some examples, the antibodies are expressed as scFvs, Fabs, scFv-Fc fusion proteins or full length immunoglobulins. In some examples, the antibodies are expressed in yeast, Escherichia coli, or mammalian cells.

In some embodiments, the method of generating renewable polyclonal antibodies specific for a target antigen further includes amplifying the target-specific polyclonal antibody population, wherein amplification of the target-specific polyclonal antibody population maintains at least 70% of the original diversity of the individual yeast clones. In some examples, the polyclonal antibody population is amplified at least 10²-fold, 10³-fold, 10⁴-fold, 10⁵-fold, 10⁶-fold, 10⁷-fold or 10⁸-fold and maintains at least 70% of the original diversity of the individual yeast clones. In particular non-limiting examples, at least 75%, at least 80% or at least 85% of the original diversity of the individual yeast clones is maintained following amplification.

In some embodiments of the method of generating renewable polyclonal antibodies specific for a target antigen, expression of the antibodies in the yeast display library is controlled by a regulatable promoter. In particular examples, the regulatable promoter is a repressible promoter. In other examples, the regulatable promoter is an inducible promoter. In some instances, the amplification step is performed under non-inducing conditions, resulting in minimal or undetectable expression of the antibodies during amplification.

In some embodiments, the polyclonal antibody population comprises at least 100, at least 150, at least 200, at least 250 or at least 300 different antibodies.

In some embodiments, the collection of antibody clones comprises an antibody display library, such as but not limited to a phage display library, a ribosome display library, a bacterial display library or an in vitro display library.

In some embodiments, the antibody display library comprises a naïve antibody library. In other embodiments, the antibody display library is derived from an immunized source, such as spleen cells or B cells obtained from an immunized human or non-human animal.

In some embodiments, the antibody display library is a single domain VH, VHH, VL, scFv or Fab library. In some examples, the antibody display library is a natural library. In other examples, the antibody display library is a synthetic antibody library. In some embodiments, the synthetic antibody library is based on a single antibody scaffold, or a limited number of antibody scaffolds. In some examples, the scaffold binds protein A and/or protein L.

In some embodiments, the collection of antibody clones comprises a phage display library, and transferring the selected phage display clones into a yeast display vector includes PCR amplification using vector encoded primers that attach homologous sequences at both ends, allowing direct cloning of the antibody (such as an scFv) into the yeast display vector.

In some embodiments, the scFv DNA from the selected yeast clones is transferred to a scFv-Fc fusion vector. Methods for transferring nucleic acid sequences to a vector are well known in the art and exemplary methods are described herein. In some examples, transfer of the yeast clone to a scFv-Fc fusion vector includes PCR amplification using vector encoded primers containing homologous sequences at both ends, allowing direct cloning of the scFv into the scFv-Fc expression vector.

In some examples of the method of the method of generating renewable polyclonal antibodies specific for a target antigen, selecting a population of antibody clones from the collection comprises at least one, at least two, at least three, at least four or at least five rounds of selection against the target antigen. In one non-limiting example, selecting a population of antibody clones from the collection comprises no more than two rounds of selection against the target antigen.

In some examples of the method of the method of generating renewable polyclonal antibodies specific for a target antigen, the collection of antibody clones comprises a phage display library and selecting the population of antibody clones from the collection comprises no more than two rounds of selection against the target antigen using phage display.

In some examples of the method of the method of generating renewable polyclonal antibodies specific for a target antigen, selecting a population of yeast clones from the yeast display antibody library comprises at least one, at least two, at least three, at least four or at least five rounds of selection against the target antigen. In one non-limiting example, selecting a population of yeast clones from the yeast display antibody library comprises no more than two rounds of selection against the target antigen.

In some embodiments, the method of generating renewable polyclonal antibodies specific for a target antigen includes providing a yeast display antibody library; selecting a population of yeast clones from the antibody library that specifically bind the target antigen to generate a polyclonal antibody population; and amplifying the polyclonal antibody population, wherein amplification of the polyclonal antibody population does not result in a significant loss in representation of individual yeast clones in the population. In some embodiments, the yeast display antibody library is generated from a population of antibody clones that was previously selected on the target antigen. In some examples, the previous selection on the target antigen comprises using an alternative display platform, such as but not limited to phage display or ribosome display.

In some examples, selection of the population of antibody clones comprises at least two, at least three, at least four or at least five rounds of selection using the alternative display platform. In some examples, the alternative display platform is selected from phage display and ribosome display. Selection is carried out according to standard methods known in the art and described herein.

In some examples, the polyclonal antibody population is amplified at least 10²-fold, 10³-fold, 10⁴-fold, 10⁵-fold, 10⁶-fold, 10⁷-fold or 10⁸-fold without significant loss in representation of the individual yeast clones.

In some embodiments, the step of selecting a population of yeast clones from the antibody library comprises at least two, at least three, at least four or at least five rounds of yeast display selection. Selection is carried out according to standard methods known in the art and described herein.

In other embodiments, the method of generating a renewable polyclonal antibody population specific for a target antigen includes providing a collection of antibody clones; selecting antibody clones from the collection that specifically bind the target antigen; transferring the selected antibody clones into a yeast display vector to generate a yeast display antibody library; selecting a population of yeast clones from the yeast display antibody library that specifically bind the target antigen to generate a polyclonal antibody population; and amplifying the polyclonal antibody population, wherein amplification of the polyclonal antibody population does not result in a significant loss in representation of the individual yeast clones. In some examples, the collection of antibody clones comprises an antibody display library. In some examples, the antibody display library comprises a phage display library or a ribosome display library. In some embodiments, the antibody display library is a scFV library. In some examples, the scFv library is a single-scaffold scFv library. In particular examples, the scaffold binds protein A and/or protein L. In some embodiments, the polyclonal antibody population comprises full-length immunoglobulins. In other embodiments, the polyclonal antibody population comprises scFv-Fc fusion proteins.

In another embodiment, provided herein is a method of generating a renewable polyclonal antibody population specific for a target antigen, wherein the method includes providing a collection of antibody clones in a first display platform; selecting from the collection a population of antibody clones that bind the target antigen to generate a first antibody display library; transferring the selected population of antibody clones into a second display platform to generate a second antibody display library; and selecting from the second antibody display library a population of clones that specifically bind the target antigen to generate a target-specific polyclonal antibody population. In some examples, the first display platform comprises phage display, yeast display, ribosome display, bacterial display or in vitro display. In some examples, the second display platform comprises phage display, yeast display, ribosome display, bacterial display or in vitro display. In one non-limiting example, the first display platform comprises phage display and the second display platform comprises yeast display.

Also provided herein are polyclonal antibodies and polyclonal antibody populations produced by any of the methods disclosed herein.

### V. Antibody Display Technologies

When animals are immunized with antigens they respond by generating a polyclonal antibody response comprised of many individual monoclonal antibody specificities. It is the sum of these individual specificities that make polyclonal antibodies useful in so many different assays. Individual monoclonal antibodies were originally isolated by immortalizing individual B cells using hybridoma technology (Kohler and Milstein, Nature 256, 495, 2011), in which B cells from an immunized animal are fused with a myeloma cell. With the advent of molecular biology, in vitro methods to generate monoclonal antibodies have been developed, in which antibody fragments, such as single chain Fvs (scFv) or Fabs, are coupled to the genes that encode them in a selectable fashion. Phage (Scott and Smith, Science 249:386, 1990; Marks et al., J Mol Biol 222:581, 1991; Bradbury and Marks, J Immunol Methods 290:29, 2004) and yeast display (Feldhaus et al., Nat Biotechnol 21:163, 2003; Boder et al., Proc Natl Acad Sci USA 97:10701, 2000; Boder and Wittrup, Nat Biotechnol 15:553, 1997) are the most common display methods. All in vitro display technologies have a number of features in common, whatever the display platform or affinity scaffold used. The general concept is that a large library of polypeptides of potential interest is created, from which ones with desirable properties can be selected. In order to carry this out a number of crucial steps are required. The first is the creation of a library at the DNA level that encodes the diversity. Antibody libraries can be created either synthetically (Hoogenboom, J. Mol. Biol. 227:381, 1992; Krebs et al., J. Immunol. Methods 254:67, 2001; Birtalan et al., Mol Biosyst 6:1186, 2010; Birtalan et al., J Mol Biol 377:1518, 2008; Fellouse et al., J Mol Biol 373:924, 2007; Sidhu and Kossiakoff, Curr Opin Chem Biol 11:347, 2007) or by harvesting natural diversity from human lymphocytes using PCR (Marks et al., J Mol Biol 222:581, 1991; Vaughan et al., Nat Biotechnol, 14:309, 1996; Lloyd et al., Protein Eng Des Sel 22:159, 2009; Schofield et al., Genome Biol 8:R254, 2007).

Once a library has been obtained at the DNA level, the next step is to couple it to the encoded antibody. This is usually carried out by cloning the library into a display vector in which the displayed protein is fused to a coat or surface protein. In the case of phage display, filamentous phage are most commonly used and the most popular display protein is g3p. In yeast display, S. cerevisiae is the preferred organism, and Aga-2 the most popular fusion partner. However, a number of less commonly used phenotype/genotype coupling methods have also been developed, including the use of protein fusion partners that bind covalently or non-covalently to specific DNA sequences (Reiersen et al., Nucleic Acids Res 33:e10, 2006; Odegrip et al., Proc Natl Acad Sci USA 101:2806, 2004; Speight et al., Chem Biol 8:951, 2001; Gates et al., J Mol Biol 255:373, 1996), and methods that directly couple RNA or RNA/DNA hybrids to the proteins they encode. In ribosome display, it is the ribosome itself that couples the nucleic acid to the encoded protein (Hanes and Pluckthum, Proc Natl Acad Sci USA 94:4937, 1997; Mattheakis et al., Proc Natl Acad Sci USA 91:9022, 1994), while in covalent display (Roberts and Szostak, Proc Natl Acad Sci USA 94:12297, 1997) a puromycin mediated covalent bond is formed between the nucleic acid and displayed protein. Naïve libraries have proved to be potent sources of antibodies against many different targets.

Once a library has been created in which the displayed protein is coupled to the gene that encodes it, the next step is to select the antibodies that bind to the target of interest. This is carried out by incubating the library with the target and separating those that bind from that do not, usually by a series of washing steps. This is followed by eluting bound antibodies, and amplifying the attached DNA that encodes them. In an ideal system, a single round of selection would be sufficient, but as enrichment is usually a maximum of 1000-fold per round, two to four selection rounds are required with amplification carried out between selections. Until recently, specific binders were identified by growing up 96-384 random clones and testing their binding properties. However, the development of deep sequencing methods has introduced a new paradigm, in which the output of selections is sequenced directly, and the most abundant clones are isolated from the library by PCR and then tested for binding (Glanville et al., Proc Natl Acad Sci USA 106:20216, 2009; Ravn et al., Nucleic Acids Res 38:e193, 2010; Di Niro et al., Nucleic Acids Res 38:e110, 2010). This has the advantage that the clones showing the greatest enrichments are tested, rather than relying on chance.

A. Phage Display

Phage display has been the most commonly used in vitro method to select antibodies in a non-therapeutic context. Depending upon the vector used, between one and five antibodies can be displayed per phage particle. Selection requires a number of cycles, each one of which involves interacting the library with the target, washing, eluting and amplifying the output, with screening usually carried out after two to three rounds. While phage display has been very successful, the selection process is essentially performed blind—until the final screens are carried out there is no indication as to whether it has been successful or not.

Most phage antibody libraries have been created by cloning large numbers of different antibody genes upstream of the gene 3 (g3) coat protein gene and using phage (Huie et al., Proc Natl Acad Sci USA 98:2682, 2001) or phagemid (Sblattero and Bradbury, Nat. Biotechnol. 18:75, 2000; Birtalan et al., J Mol Biol 377:1518, 2008; Vaughan et al., Nat Biotechnol 14:309, 1996; de Haard et al., J Biol Chem 274:18218, 1999; Sheets et al., Proc. Natl. Acad. Sci. U.S.A. 95:6157, 1998; Knappik et al., J. Mol. Biol. 296:57, 2000; Gilbreth et al., J Mol Biol 381:407, 2008) as the display vehicles. In general, phagemid vectors are preferred as cloning is more straightforward and they are more stable. However, whereas display using phage is multivalent (up to five copies), using phagemids is monovalent, with only 1% of phagemid particles actually displaying antibodies (Clackson and Wells, Trends Biotechnol 12:173, 1994). Recently a number of helper phage systems (Kramer et al., Nucleic Acids Res 31:e59, 2003; Soltes et al., J Immunol Methods 274:233, 2003; Rondot et al., Nat Biotechnol 19:75, 2001; Baek et al., Nucleic Acids Res 30:e18, 2002; Duenas and Borrebaeck, FEMS Microbiol Lett 125:317, 1995) have been described in which g3 is either deleted or inactivated, resulting in multivalent display. However, they can be difficult to use because of the difficulty in obtaining high titers of the helper phage. An alternative system based on the concept of packaging cells hosting helper plasmids (Chasteen et al., Nucleic Acids Res 34:e145, 2006) was developed, providing the flexibility of either monovalent or multivalent display from the same library, without the need for helper phage.

Most antibody libraries use scFvs (Huston et al., Proc Natl Acad Sci USA 85:5879, 1988) as the antibody format, in which a flexible linker joins the VH and VL genes or Fabs, in which VH-CH1 and VL-CL associate non-covalently. The antibody genes are derived either from natural sources (e.g. human peripheral blood lymphocytes) or created synthetically by introducing diversity using oligonucleotides into frameworks with desirable properties. Originally completely degenerate oligonucleotides encoding all 20 amino acids were used to generate diversity (Hoogenboom and Winter, J. Mol. Biol. 227: 381, 1992; Krebs et al., J. Immunol. Methods 254:67, 2001). However, more recently it has been found that the use of diversity restricted to only three or four amino acids can provide antibodies with similarly high affinities (Birtalan et al., Mol Biosyst 6:1186, 2010; Birtalan et al., J Mol Biol 377:1518, 2008; Fellouse et al., J Mol Biol 373:924, 2007; Sidhu and Kossiakoff, Curr Opin Chem Biol 11:347, 2007).

When an antibody gene is cloned upstream of gene 3, the antibody is displayed as a fusion protein with the gene 3 coat protein. A library of such phage antibodies theoretically consists of as many as 10¹¹ different members (the diversity is usually measured by counting the number of independent colonies), with each different specificity being represented by a relatively small number of phage in a library. In general, diversity is limited by the transfection efficiency of bacteria, and the largest libraries (Vaughan et al., Nat Biotechnol, 14:309, 1996; Lloyd et al., Protein Eng Des Sel 22:159, 2009; Schofield et al., Genome Biol 8:R254, 2007; Rothe et al., J Mol Biol 376:1182, 2008) require hundreds or thousands of electroporations to generate the required diversity.

A recombinatorial method of library creation was developed (Sblattero and Bradbury, Nat. Biotechnol. 18:75, 2000), in which the VH/VL linker contains a translated lox recombinase site. This allows VH and VL genes from two different scFvs to be exchanged by recombination in the presence of Cre recombinase. For example, if two scFvs VH1-VL1 and VH2-VL2 are recombined, the products of recombination are VH1-VL1, VH1-VL2, VH2-VL1 and VH2-VL2, with all scFvs being functional. By superinfecting E. coli with at least 20 different phagemid antibodies, it was shown that extensive recombination occurred between different VH and VL genes, with each individual bacterium producing as many as 400 different antibodies (Sblattero and Bradbury, Nat. Biotechnol. 18:75, 2000). Extremely large diversity libraries can be made using this method, without the need to carry out countless transformations. Furthermore, when the library is created by recombination, it is simultaneously amplified, avoiding the bias that occurs when traditional libraries are amplified by growth. As a result, there is enough primary library to carry out over one million selections, more than adequate to carry out the genomic scale selections described herein.

B. Yeast Display

Whereas in phage display no more than 5 antibodies can be displayed per phage particle, this increases dramatically to approximately 30,000 antibodies in yeast (Boder and Wittrup, Nat Biotechnol, 15:553, 1997). This coupled with the size of yeast, results in significant advantages as well as disadvantages. The main advantage is the ability to use flow cytometry to both analyze and sort library selections. Two different fluorescent dyes are usually used. One reflects the amount of antibody displayed, while the other the amount of antigen bound. This provides great flexibility and immediate feedback on the progress of a selection, unlike phage display. Furthermore, sorting higher affinity clones is far easier than with phage display. By normalizing to antibody display levels, antibodies with higher affinities, rather than greater expression levels can easily be selected. In fact, it is possible to distinguish and sort antibodies whose affinities differ by only two-fold (VanAntwerp and Wittrup, Biotechnol Prog 16:31, 2000).

As large affinity improvements are usually caused by many small additive improvements, yeast display is ideally suited for affinity maturation, and holds the record for the antibody with the lowest evolved affinity: 48 fM (Boder et al., Proc Natl Acad Sci USA 97: 10701, 2000). These advantages are offset by the disadvantages associated with the creation and sorting of large naïve libraries. Although a large naïve scFv yeast display library has been made (de Bruin et al., Nat Biotechnol 17, 397, 1999), the volumes of library required (>10 ml) for each selection is enormous. Not only does this result in significant up-front work to create usable library aliquots, but also the relatively slow pace (10,000-30,000 events per second) of fluorescence activated cell sorting (FACS) makes it impossible to use with naïve libraries unless prior pre-selection has been carried out. This makes yeast display ideal for affinity or specificity maturation where the library size is reduced to ˜10⁶ or less. Direct comparisons between libraries created from the same source of HIV immune DNA indicate that when selecting scFvs, antibody diversity was greater from a yeast display library than from a phage display library (Bowley, D. R. Yeast and phage display of antibodies: comparisons and new developments Ph.D. thesis, The Scripps Research Institute, 2007). However, antibodies from HIV immune sources tend to be highly mutated, and these experiments have not been repeated with naïve libraries.

C. Exploiting the Recombinant Nature of In Vitro Antibodies

All in vitro selection systems immediately provide the genes, and corresponding sequences, of antibodies selected against a particular target, providing ready access to additional antibody formats by simple sub-cloning, greatly broadening the utility of antibodies following selection. Functions adopted using this “gene-based” approach include dimerization (de Kruif and Logtenberg, J Biol Chem 271, 7630, 1996), multimerization (Hudson and Kortt, J Immunol Methods 231, 177, 1999; Dübel et al, J Immunol Methods 178, 201, 1995), and fusions to enzymes (Griep et al, Protein Expr Purif 16, 63, 1999), tags (Cloutier et al, Mol Immunol 37, 1067, 2000) or fluorescent proteins (Casey et al, Protein Eng 13, 445, 2000). In the case of scFvs, bivalent dimers (diabodies) (Perisic et al, Structure 2, 1217, 1994), trimers (Atwell et al, Protein Eng 12, 597, 1999; Pei et al, Proc Natl Acad Sci USA 94, 9637, 1997) and even tetramers (Le Gall et al, FEBS Lett 453, 164, 1999) can be formed when the VH/VL linker is shortened. Fusion to alkaline phosphatase is a particularly useful example of improved functionality. As this is a dimeric enzyme, fusing antibodies, either individually or as libraries, to alkaline phosphatase simultaneously provides dimerization and alkaline phosphatase activity, greatly facilitating screening in high throughput contexts (Schofield et al., Genome Biol 8, R254, 2007; Griep et al, Protein Expr Purif 16, 63, 1999). However, each of these approaches fuses only a single functionality to each antibody, and new constructs must be created for each additional functionality. In order to overcome this, two general approaches have also been developed. In the first, fusion of a short peptide in vivo biotinylation tag (Cloutier et al, Mol Immunol 37, 1067, 2000) to the carboxy terminus allows stoichiometrically defined site specific antibody biotinylation. This provides multimerization (Thie et al, N Biotechnol, 26, 314, 2009) as well as access to the broad range of commercially available streptavidin derivatives.

A second method we developed is based on the fusion of an E. coli to the C terminus of the antibody (Ayriss et al., Methods Mol Biol 525, 241, 2009; Ayriss et al, J Proteome Res 6, 1072, 2007). This is a 35 amino acid peptide that binds to its partner K-coil with an affinity of 60 pM (De Crescenzo et al, 1754, 2003), allowing the antibody to be specifically labeled with any other target that has a K-coil fused to it. In addition to using this to bind K-coil modified GFP and alkaline phosphatase, it has also been shown that scFvs can be labeled with chemically synthesized Alexa 488 K-coil peptides (Ayriss et al., Methods Mol Biol 525, 241, 2009; Ayriss et al, J Proteome Res 6, 1072, 2007). The availability of recombinant binding domains also allows the creation of bispecific antibodies using a number of different methods (see Muller and Kontermann, BioDrugs 24, 89, 2010 for a review). Bispecific antibodies bind to two targets. Although the predominant use for this has been in the realm of therapeutics, the creation of bispecific scFvs that recognize different epitopes of the same target can result in enormous affinity increases without the need for mutation and further selection (Neri et al, J Mol Biol 246, 367, 1995).

Antibody fragments can additionally be transformed into full-length antibodies (Persic et al, Gene 187, 9, 1997), although this can be relatively time-consuming due to the need to separately clone the VH and VL domains. This can be overcome if Fab libraries in carefully designed phage display vectors are used in which the VH/CH1 and VL/CL domains can be recloned as single fragments into a compatible mammalian expression vector (Jostock et al, J Immunol Methods 289, 65, 2004). A more straightforward alternative is the creation of scFv-Fc fusions (Shu et al, Proc Natl Acad Sci USA 90, 7995, 1993). These are very similar to full-length antibodies in many aspects, including their recognition by secondary reagents and biological properties (Powers et al., J. Immunol. Methods 251, 123, 2001; Shu et al, Proc Natl Acad Sci USA 90, 7995, 1993; Brocks et al, Immunotechnology 3, 173, 1997; Hayden et al, Ther Immunol 1, 3, 1994). However, they avoid the need for balanced production of heavy and light chains, requiring only a single chain to be produced, and have been functionally expressed in P. pastoris (Powers et al., J. Immunol. Methods 251, 123, 2001; Ren et al, Biotechnol Lett 30, 1075, 2008; Liu et al, J Biochem Tokyo 134, 911, 2003), mammalian cells (Shu et al, Proc Natl Acad Sci USA 90, 7995, 1993), plant seeds (Van Droogenbroeck et al, Methods Mol Biol 483, 89, 2009) transgenic quail (Kawabe et al, J Biosci Bioeng 102, 297, 2006), and as inclusion bodies in E. coli, from which functional scFv-Fc fusions could be refolded (Cao et al, Appl Microbiol Biotechnol 73, 151, 2006). scFv-Fc fusions have been expressed in S. cerevisiae, (see Examples below) as have full length antibodies (Rakestraw and Wittrup, Biotechnol Bioeng 93, 896, 2006; Rakestraw et al, Biotechnol Bioeng 103, 1192, 2009; Horwitz et al, Proc Natl Acad Sci USA 85, 8678, 1988).

Microinjected antibodies have been long used to knock out intracellular functions (Gawlitta et al, Eur J Cell Biol 26, 83, 1981). Antibody fragments can be expressed within target cells and targeted to various subcellular compartments (Persic et al, Gene 187, 9, 1997; Kontermann, Methods 34, 163, 2004) by adding suitable signal sequences, allowing visualization or functional modification of proteins in different compartments, in a process termed “intracellular immunization” (Biocca and Cattaneo, Trends Cell Biol 5, 248, 1995). Removing the standard leader sequence results in cytoplasmic expression while the addition of a nuclear localization signal targets to the nucleus. The combination of a leader sequence and the endoplasmic reticulum (ER) retention sequence retains expressed antibodies in the ER and has been used to prevent the expression of membrane proteins by sequestration in the ER (Beerli et al, J Biol Chem 269, 23931, 1994; Richardson et al, Proc Natl Acad Sci USA 92, 3137, 1995; Paganetti et al, J Cell Biol 168, 863, 2005; Strebe et al, J Immunol Methods 341, 30, 2009). The advantage of this strategy is that it requires antibodies that bind to any accessible epitope to provide the functional knockout, as opposed to the functional activity required of cytoplasmically expressed antibodies. Functional studies of membrane receptors or secreted proteins can thus be attempted by a single standardized subcloning step immediately after in vitro antibody selection, providing equivalence to RNAi knockdowns at the protein level. While expression in the secretory pathway is straightforward, folding of antibody fragments in the cytoplasm is far more challenging, due to the absence of specific chaperones, and the reducing environment, which prevents disulfide bond formation (Biocca et al, Bio/Tech 13, 1110, 1995). Despite these problems, there are examples where cytoplasmic proteins have been targeted with intracellular scFvs (Biocca et al, BioTech 12, 396, 1994; Nizak et al, Science 300, 984, 2003). The success of this approach has been improved by the creation of libraries of particularly stable scFvs (Desiderio et al, J Mol Biol 310, 603, 2001; der Maur et al, J Biol Chem 277, 45075, 2002; Tanaka et al, Nucleic Acids Res 31, e23, 2003), preselecting antibodies for functional cytoplasmic expression (Auf der Maur et al, FEBS Lett 508, 407, 2001; Visintin et al, Proc Natl Acad Sci USA 96, 11723, 1999), or by using binder libraries based on molecular scaffolds that do not rely on disulfide bond formation, such as engineered ankyrin repeat proteins (Amstutz et al, J Biol Chem 280, 24715, 2005; Kohl et al, Structure Camb 13, 1131, 2005). One major advantage of such protein based allosteric blockers is the ability to generate very specific binders, able to distinguish between closely related family members, or different conformations of the same protein. While the need to genetically modify the target cell is a disadvantage, this has been partly alleviated by fusion to internalizing sequences that allow antibodies to enter the cell from the outside (Rizk et al, Proc Natl Acad Sci USA 106, 11011, 2009).

D. High Throughput Selection by In Vitro Display Methods

The ease with which antibodies can be selected, screened and produced by in vitro display technologies, makes generation and screening of antibodies rapid and relatively straightforward compared to hybridomas. Typically a panel of ELISA positive monoclonal recombinant antibody fragments can be generated against a single target within a couple of weeks. Early experiments demonstrated the feasibility of semi-automated selection/screening of phage antibody libraries (Lou et al., J. Immunol. Methods 253, 233, 2001; Hallborn and Carlsson, Biotechniques Suppl, 30, 2002; Turunen et al, J Biomol Screen 14, 282, 2009) on small numbers of targets. These included the use of robotics (Hallborn and Carlsson, Biotechniques Suppl, 30, 2002), and selection in the microtiter format, using antigen immobilized on pins (Lou et al., J. Immunol. Methods 253, 233, 2001) or on magnetic beads (Turunen et al, J Biomol Screen 14, 282, 2009). Recently, selections on over 400 different antigens were successful with 72% of targets yielding antibodies (Schofield et al., Genome Biol 8, R254, 2007).

In a recent international comparative study, monoclonal recombinant antibodies were raised to 20 different human SH2 domains using hybridomas or phage display. Phage display was able to generate antibodies (scFvs and Fabs, some with sub-nanomolar affinities) against all the targets (Mersmann et al, N Biotechnol, 27, 118, 2009; Pershad et al, Protein Eng Des Sel 23, 279, 2010), while hybridomas failed for two antigens. Only phage display was able to generate antibodies specific for ABL1 or ABL2, which differ from each other by 11%. Furthermore, 1920 hybridomas were screened for each antigen, while the number of phage antibodies screened ranged from 24 to 190, depending upon the laboratory. These antibodies were validated in a broad range of assays, with some functional in microarrays, immunoblots, immunofluorescence and immunoprecipitation. In all these studies, the final antibody format was monoclonal scFv or Fab fragments, indicating that high throughput selection using phage display is feasible. Positive antibody clones were identified by enzyme linked immunosorbent assays (ELISAs), which tends to use significant amounts of antigen, especially if extensive screening is carried out.

One commonly cited issue regarding phage antibodies relates to the ability to express them. In general, the expression and stability of antibody fragments varies enormously, from exceptionally stable scFv fragments used in clinical trials (Kreitman, et al, J Clin Oncol 18, 1622, 2000) to other fragments that are poorly expressed. Selection for stability and expression can be included as part of the high throughput screening process, and libraries based on stable scaffolds tend to produce antibodies that are more stable and well expressed (Fellouse et al., J Mol Biol 373, 924, 2007). As soon as a monoclonal antibody fragment is generated, it can be defined precisely by its VH and VL sequence, and even “distributed” in this way.

The following examples are provided to illustrate certain particular features and/or embodiments. These examples should not be construed to limit the disclosure to the particular features or embodiments described.

### EXAMPLES

### Example 1

### Yeast Display

Yeast display was first used to improve the affinities and specificities of humanized antibodies or ones selected by phage display. This was carried out by creating error prone libraries within the context of a yeast display vector designed to be compatible with the phage display vector (Sblattero and Bradbury, Nat. Biotechnol. 18, 75, 2000). This is best illustrated by a pair of antibodies we selected from the phage antibody library to distinguish between phosphatidylinositol phosphate 345 (PIP345) and phosphatidylinositol phosphate 45 (PIP45), two signaling compounds differing by a single phosphate. Error prone libraries were made of both antibodies, displayed on yeast and further selected for improved binding to the recognized PIP. Improved clones showed 5-10 fold increase in binding to their cognate PIP. In the case of clone A12 (recognizing PIP45), simultaneous negative selection against PIP345 also resulted in improved discrimination between the two PIPs.

With traditional phage display, as described above, 96 clones are usually screened for binding after selection has been carried out. In separate experiments using deep sequencing to identify protein interaction partners (Di Niro et al., Nucleic Acids Res 38, e110, 2010), it was found that screening only 96 clones after selection missed most interaction partners, and in one of the selections, over 1000 clones would have to be screened to have a 90% chance of identifying the top ten sequenced clones. Given that during phage antibody selection the starting diversity is far greater, the first round output is usually 10⁴⁻⁶ and the percentage of positive antibodies after one round is 0.6-3%, many of which are diverse (de Wildt et al., Nat. Biotechnol. 18, 989, 2000), it is likely that even more positive clones are missed during antibody selection if only 96 are screened. This has been confirmed by recent deep sequencing of antibody selections from synthetic libraries (Ravn et al., Nucleic Acids Res 38, e193, 2010), in which it was discovered that if 96 clones were screened, seven of the top ten scFvs (ranked by their sequenced frequency), all of which bound antigen, were not identified. In other words, the traditional ELISA based method of screening phage antibody outputs does not identify most of the top antibodies.

The size of yeast display libraries that can be easily cloned is 10⁵⁻⁶, very similar to the first round output of phage antibody selections. Consequently, it would appear that using yeast display to screen the full output of a selection would be a far more efficient and comprehensive screening method: equivalent to carrying out up to a million ELISAs, rather than the usual 96. Furthermore, this approach may give two additional advantages. Given that proteins that are well displayed on yeast tend to be more thermostable and better expressed as soluble proteins (Shusta et al., J. Mol. Biol. 292, 949, 1999), this process should also favor more stable well expressed antibodies, desirable properties within the context of a high throughput antibody selection pipeline. In preliminary experiments, ELISA signals of scFvs generated by traditional phage display were compared with scFvs generated by a first round of phage selection followed by selection using yeast display. Those selected using yeast show higher ELISA signals, as well as higher expression levels (in E. Coli). It is well known that the more selection rounds carried out using phage, the more restricted diversity becomes. Interestingly, antibodies that are lost in later rounds are not necessarily the lower affinity ones, high affinity antibodies are also lost (Ravn et al., Nucleic Acids Res 38, e193, 2010). By combining phage and yeast display, it is expected that the full diversity of the specific antibody binding diversity can be selected.

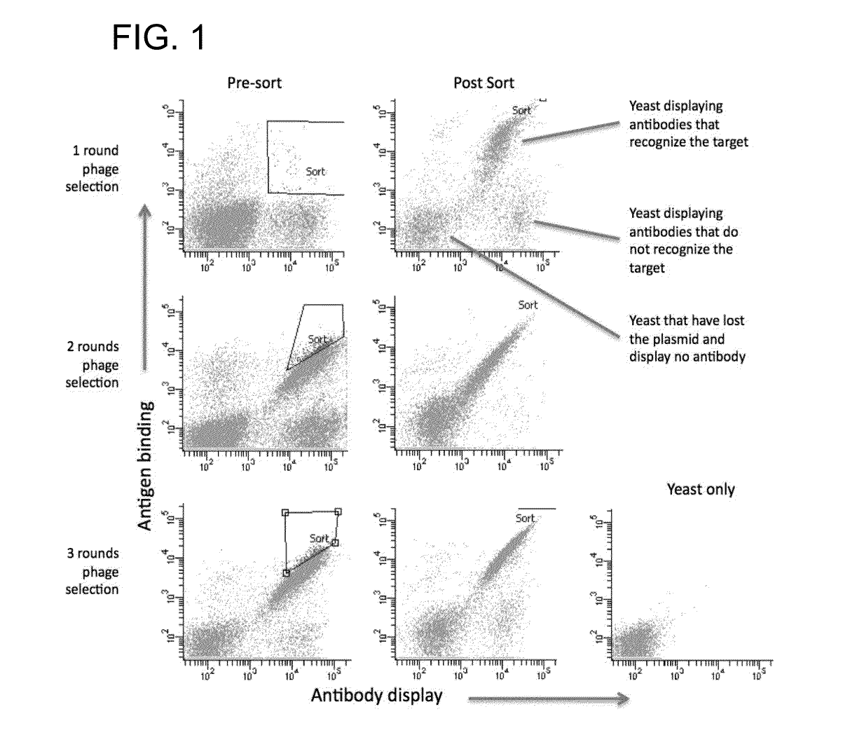

In order to study the advantages of combining phage and yeast display, one, two or three rounds of phage antibody selection were carried out against the IgE receptor and the output was cloned into the compatible yeast display vector (FIG. 1). The population in the upper right quadrant in each plot represents those yeast clones that display antibody and bind to the target, while clones in the bottom right, display antibody, but do not bind target. The population in the lower left quadrant has lost the plasmid and does not display any antibody. In the case of the library cloned after a single round of phage selection, most clones displaying antibody do not recognize the target. However, a single round of florescence activated cell sorting (FACS) is sufficient to render the majority target-specific, with 56% of tested monoclonals recognizing the target. This is in contrast to libraries cloned after two or three rounds of phage selection, which are already predominantly target specific, with 92% and 100% of monoclonals being target specific, respectively, after an additional round of FACS. This would seem to suggest that two rounds of selection, phage or yeast, are required. However, a careful examination of the shape of the population distribution in the three cases suggests that the most diverse population is likely to be that selected after one round of phage display, as demonstrated by the broader and less uniformly diagonal shape.

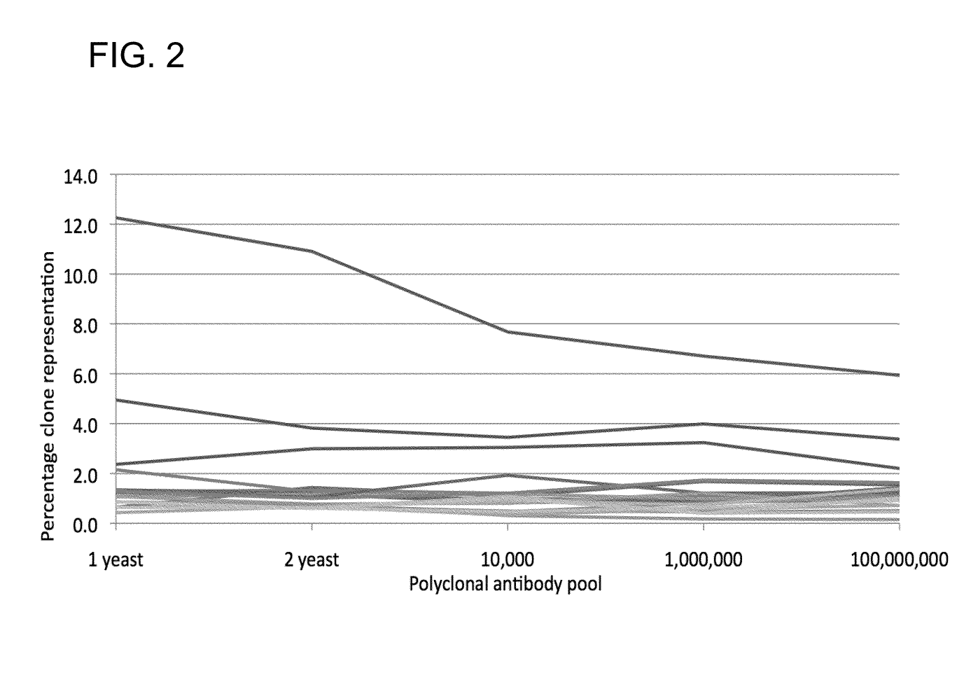

454 sequencing was carried out on the polyclonal population obtained after one round of phage and one round of yeast display selection (equivalent to the population shown in FIG. 1, top right—termed yeast 1), as well as after an additional round of yeast selection (yeast 2). This identified >500 different antibody VH sequences (each defined as a group with ≧95% homology), of which the most abundant represented ˜11% after two rounds of yeast selection. This population was amplified 10⁸ times (four overnight growths of 100 fold dilution each) and re-sequenced after each overnight growth. Remarkably, the occurrence of each of the top 20 VH sequences remains astonishingly constant, as shown in FIG. 2. In fact, 82% of the top 100 sequences had a less than three-fold (and 62% less than two-fold) variation in their frequency after 10⁸ fold amplification. These results demonstrate that growth of yeast under repressive conditions results in minimal amplification bias, and that recombinant polyclonal antibodies propagated under these conditions can be considered infinitely renewable (1 ml to 100,000 liters).

### Example 2

### scFv-Fc Fusions

scFv-Fc fusion vector derivatives were created (Di Niro et al., BMC Biotechnol 7, 46, 2007) based on original immunoglobulin expression vectors (Persic et al, Gene 187, 9, 1997). These have been expressed in vivo as well as in tissue culture cells (Di Niro et al., BMC Biotechnol 7, 46, 2007). A vector that drives the expression of scFv-Fc fusions in P. pastoris was also created, similar to those created by others (Powers et al., J. Immunol. Methods 251, 123, 2001; Ren et al, Biotechnol Lett 30, 1075, 2008; Liu et al, J Biochem Tokyo 134, 911, 2003), as well as a vector for expression in S. cerevisiae. It was found that the expression levels in S. cerevisiae were similar (˜50 mg/L) when expressed in the strain YVH10 (Rakestraw et al, Biotechnol Bioeng 103, 1192, 2009), which co-expresses yeast protein disulfide isomerase, to those in P. pastoris (50-60 mg/L). ELISA signals for antibodies produced in the two systems were equivalent at similar concentrations.

### Example 3

### Selection of a Stable scFv Scaffold

The library was screened (Sblattero and Bradbury, Nat. Biotechnol. 18, 75, 2000) for a scFv that bound to protein A and protein L. These two bacterial super-antigens recognize conformational epitopes (Graille et al., Proc. Natl. Acad. Sci. U.S.A. 97, 5399, 2000; Graille et al., Structure (Camb) 9, 679, 2001; Wikstrom et al., J Mol Biol 250, 128, 1995) and can therefore be used to identify and select correctly folded VH and VL domains respectively. The result of this selection and screening was E7, an scFv that bound strongly to both protein A and protein L, and demonstrated remarkable stability, shown by the ability to remain folded in 200 mM DTT at 40° C. for an hour, conditions under which many scFvs unfold as a result of reduction of the stabilizing disulfide bonds. Most scFvs are non-functional if expressed and folded in the cytoplasm, and require secretion into the periplasm (if expressed in E. coli) or the secretory compartment of eukaryotic cells to be active. E7, by contrast, when expressed in the E. coli cytoplasm remains fully active, continuing to bind protein A and L.

### Example 4

### Antibody Selection Pipeline

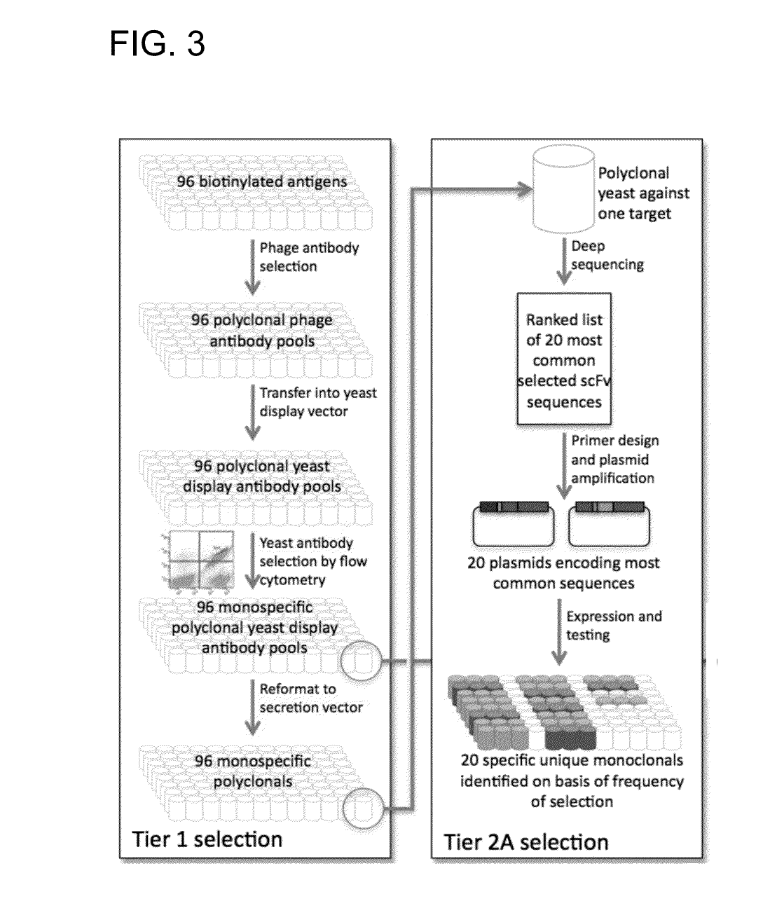

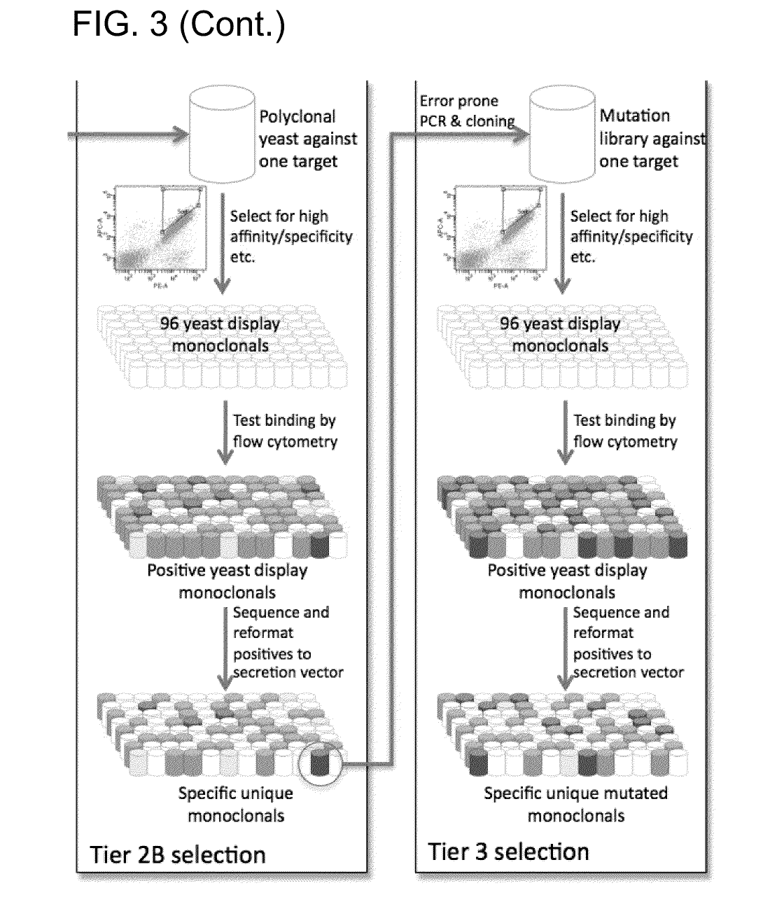

The selection of high quality antibodies requires high quality targets. Where possible, targets produced by specialized protein expression and purification centers (e.g. the SGC or soluble PrESTs produced by the Human Protein Atlas) will be used. Proteins will be quality controlled upon arrival, and prior to use for selection. The antibody selection pipeline will be predominantly established using these validated targets. However, expertise with GFP folding reporters will also be used to create a target expression pipeline that will (1) assess clones for their solubility; and (2) select clones encoding soluble protein fragments from genes that express insoluble proteins. In addition to using soluble proteins, the use of virus like particles (VLP) displaying membrane proteins will also be assessed. The antibody selection pipeline will encompass selection, characterization and preliminary production within the context of the three-tier philosophy described above and illustrated in FIG. 3.

### High-Throughput Pipeline for Target Quality Control and Generation

Received protein targets will be assessed for purity, solubility, correct size and concentration by PAGE initially, transitioning to microfluidic approaches (e.g. LabChip GXII). As most specialized centers carry out quality control, including mass spectrometry, this QC will be predominantly confirmatory. The targets from the SGC will be provided already biotinylated. PrEST targets will be assessed for solubility upon receipt and biotinylated using commercial kits (Innova). After biotinylation PrESTs will be assessed by PAGE.

### Target Production Vector

A multifunctional vector will also be used. The N-terminal AviTag is a short peptide that can be biotinylated in vivo (Ashraf et al., Protein Expr Purif 33, 238, 2004; Schatz, Biotechnology (NY) 11, 1138, 1993), and will be used to purify and immobilize the expressed target on magnetic streptavidin beads for phage antibody selection. Following the AviTag, is the split GFP11 tag. This will be used for the initial solubility screening: only if the target is soluble will the GFP11 be available to complement the GFP1-10 fragment to produce fluorescence (Cabantous and Waldo, Nat Methods 3, 845, 2006; Cabantous et al., Nat Biotechnol 23, 102, 2005; Cabantous et al., J Struct Funct Genomics 6, 113, 2005). This tag will also be used to fluorescently label the target during yeast display selection and may eliminate the need to purify target. The tobacco etch virus (TEV) protease (Kapust et al., Biochem Biophys Res Commun 294, 949, 2002) is between the GFP11 and the target. After phage antibodies have bound to the streptavidin-immobilized target, TEV protease will be used to release the target, and attached binding phage, from the streptavidin beads. This approach (Wrighton et al., Science 273, 458, 1996) has the advantage that only specific phage binding to the target are eluted, and not those binding nonspecifically to streptavidin or beads. The 6H is tag will be used for target purification if required. This vector will initially be tested using PrEST clones from the HPA. It has been found that PrEST solubility is an important factor in the successful selection of antibodies using phage display (Gustaysson et al., N Biotechnol, 28, 302, 2011), by using this vector it will be possible to simultaneously screen, express and purify soluble target PrESTs.

### High-Throughput Solubility Screening of Candidate Target Clones

Target clones, both full length and PrESTs, will be cloned into expression vector using PCR and one-step isothermal in vitro recombination (Gibson et al., Nat Methods 6, 343, 2009). PrEST clones will be obtained from HPA, while full length cDNA clones will initially be obtained from DNASU (online at dnasu.asu.edu/DNASU/Home.jsp). Once the pipeline is in production mode, open reading frame (ORF) clones will be obtained, which will be usable with generic primer pairs.

PCR products will contain terminal sequences overlapping with the vector, allowing rapid enzymatic cloning (Gibson et al., Nat Methods 6, 343, 2009). It has been found that this process allows high-throughput sample processing without restriction digestion and product purification. Clones will be sequenced. Target solubility will be determined using the high throughput split GFP solubility assay (Listwan et al., J Struct Funct Genomics 10, 47, 2009) and soluble clones taken forward for use in antibody selections. Proteins will be induced and expressed in the third generation GNF robot (Lesley, Protein Expr. Purif 22, 159, 2001), which is able to express and purify protein from the equivalent of 96 one liter cultures. For protein targets that fail the solubility test, a pilot pipeline will be developed that will fragment each gene and test the encoded random polypeptide fragments for improved solubility. Full-length PCR products will be fragmented by acoustic focusing the Covaris system. This provides very tight length distributions, avoiding the need for gel purifications, and is widely used in sequencing centers (Quail et al., Nat Methods 5, 1005, 2008). After end repair, fragmented DNA will be cloned into the expression/solubility screen vector. Each library will be plated out using the liquid handling robot, and fluorescent clones picked after induction (Cabantous et al., Nat Biotechnol 23, 102, 2005) using the Glowpix robot. Clones will be assayed for soluble protein production in liquid cultures, sequenced and optima used as candidates for antigen production. The overall goal of this pipeline is for each insoluble target to provide at least one, but preferably 2-3, soluble fragments usable as selection antigens with a final planned throughput of 96 per week.

### Phage Antibody Library Production and Quality Control Pipeline

One advantage of natural antibody libraries is that the great diversity of V regions allows the selection of antibodies against many different targets, such as the tyrosine sulfate (Kehoe et al., Mol Cell Proteomics 5, 2350, 2006) and phosphatidylinositol phosphate antibodies described herein. However, difficulties are sometimes encountered expressing some selected scFvs from this library. While this is mitigated to some degree by carrying out selection in yeast after phage display (which tends to select for clones that are better expressed), preliminary results suggest that this may be less of a problem when scFvs are expressed as scFv-Fc fusions. There are advantages to the use of libraries based on a single well-expressed stable scaffold, rather than diverse natural libraries, especially if a single antigen class, such as proteins, is to be targeted. These include increased expression levels, greater stability and the use of common primers for amplification and sequencing. In addition, restricted diversity in a single scaffold allows the use of next generation sequencing platforms with shorter reads. Furthermore, a single scaffold makes informatic antibody distribution more straightforward, as specificities can be recreated using only oligonucleotides encoding CDRs. When the scaffold binds to protein A and L, these reagents can also be used for detection, purification and assessment of correct folding.

The selection pipeline will be established using the present library, while single scaffold libraries are developed. While single scaffold libraries have the advantages described above, ultimately, it will be antibody quality (affinity, diversity and expression levels) that determines whether one or both are used routinely. Whichever library is used, it is important to establish production and QC criteria for phage antibody libraries to be used for selection, as it has been found that antibody quality depends crucially on library quality, best assessed by western blotting to determine scFv display levels. Library can be produced in large shake flasks or using the GNF robot (Lesley, Protein Expr Purif 22, 159, 2001), which has the potential to increase library production capacity by ˜100 fold, by virtue of high density culture using sparged air/O₂.

A new scFv library will be created using the stable E7 scaffold described above. This will be created using trinucleotide primers (Trilink Inc.) based on those previously described (Birtalan et al., J Mol Biol 377, 1518, 2008), in which the predominant diversity (used for HCDR1, HCDR3, LCDR1 and LCDR3) will initially be tyrosine, serine, glycine and alanine (25, 20, 20 and 15% each respectively) with 5% phenylalanine, tryptophan, histidine, proline and valine, with significant length variation in both CDR3s. CDR2 diversity will be more restricted. This library will be created using Kunkel mutagenesis (Kunkel, Proc. Natl. Acad. Sci. U.S.A. 82, 488, 1985) into pDAN5, a lox recombination vector (Sblattero and Bradbury, Nat. Biotechnol. 18:75, 2000), allowing the use of Cre-based recombination to increase library diversity, as well as to increase the amounts of available library. The performance of both libraries (natural and E7 scFv) will be assessed by selection against a panel of antigens using the proposed pipeline, and determining affinity, diversity (assessed by deep sequencing) and expression levels for selected antibodies. Depending upon the sequences of selected antibodies new libraries comprising improved diversity will be built and tested.

High Throughput Antibody Selection Pipeline for scFv-Fc Fusions

This specific aim comprises the implementation of the selection platform, as well as the subsequent production of scFv-Fc fusions, including the creation of an optimal expression vector. The selection scheme is illustrated in FIG. 3. Selections have been carried out using combined phage and yeast display for many targets at the single target scale, with flow cytometry carried out target-by-target using the FACSAria flow cytometer. A transition from the single target to 96 targets simultaneously in the microtiter format will be made. This will require each well of the microtiter plate to correspond to a single target as shown in FIG. 3. The individual steps and how they will be automated are described below. This includes cloning from phage to yeast display vectors, and subsequently from yeast display to yeast secretion vectors. Assessment of antibody diversity will be carried out by deep sequencing as described below.

### Phage Selection

Phage selection has been automated at the 96 well scale by adapting the Kingfisher Flex magnetic particle processor for this purpose. Approximately 1 μg of biotinylated antigen is incubated with 10¹²⁻¹³ phagemid antibody particles. Streptavidin coated magnetic beads are added to each well and sequentially transferred to different wash buffers, and eventually an elution buffer. Eluted phagemid particles are isolated and reinfected into E. coli for continued phage selection, or PCR amplified for recloning into yeast display vectors.

The target format described above allows incorporation of additional specificity into the selection. Each target will be tagged with biotin, GFP11 and a TEV protease site at the N terminus and a His6 tag at the C terminus, allowing for the use of the already developed Kingfisher protocols, with the modification that elution will be carried out with TEV protease. This will release the target from the streptavidin-coated beads, ensuring that only phage bound to the target are eluted. A series of helper plasmids providing multivalent display were developed (Chasteen et al., Nucleic Acids Res 34, e145, 2006), that have been used in phage antibody selections. One advantage of using these in combination with yeast display is that a multivalent first round selection is likely to harvest greater diversity for subsequent yeast based selection than standard helper phage. It is expected that one or two rounds of phage selection will be used prior to yeast display: one round requires longer yeast sorting, while two rounds may have reduced diversity (FIG. 1). The relative advantages of each will be determined by assessing affinity, specificity and diversity.

Cloning of Phage Selection Outputs into Yeast Display Vector

First round phage selections will be amplified by PCR using vector encoded primers that attach homologous sequences at both ends, allowing direct cloning of the scFv into the yeast display vector by homologous recombination. This will be carried out on our robotic platform using the integrated TETRAD PCR machine. The resulting PCR products will be analyzed using the eGENE microtiter microfluidic platform to confirm full length amplification and determine concentration, and mixed with the digested yeast display vector for in vivo recombination following transformation into chemically competent yeast cells in the 96 well format. Following cloning of 96 phage selections into the yeast display vector, at least 10⁵ clones per target will be the aim. This will be determined by dilution and plating of the transformations using the liquid handling robot onto OMNI trays with 96 individual zones. Concurrently, after transformation, the remainder of the transformed yeast cells will be grown up in liquid for subsequent sorting.

### Optimization of Yeast Display Selection

Selection of yeast clones using the FACSAria is relatively straightforward for single clones. The antigen is usually biotinylated, incubated with yeast and labeled with streptavidin phycoerythrin. However, a different approach will be explored using the target format proposed above, in which the split GFP (Listwan et al., J Struct Funct Genomics 10, 47, 2009; Cabantous and Waldo, Nat Methods 3, 845, 2006; Cabantous et al., Nat Biotechnol 23, 102, 2005) will be used. The target will be tagged with the 15 amino acid GFP11 fragment. Both this and the complementary GFP1-10 fragment are non-fluorescent unless they are mixed together, whereupon a fluorescent GFP is reconstituted. After reconstitution, fluorescence is directly proportional to molar target concentration. This allows the split GFP to be used to first identify soluble clones as described above, and subsequently for target labeling during flow cytometric selection as described here. This provides the additional advantage that antigen may not need to be purified, as only reconstituted GFP associated with the target will be fluorescent and able to fluorescently label the yeast, a possibility that will be explored. Should split GFP labeling be ineffective, fluorescent streptavidin will be used to label the in vivo added biotin.

Sort gates are based on antigen binding and display levels, with clones in the upper right quadrant (see FIG. 1) being selected. When carried out at the microtiter plate scale, the goal will be to sort binding clones from one well (e.g. A1) in the origin plate into the same well (A1) of the destination plate. Depending upon the number of phage selection cycles carried out, and consequently the percentage of positive clones, sorting for each target will either require 1-2 hours for libraries which have undergone one round of phage selection (to obtain 10⁶ clones), or 5-10 minutes for those after two rounds of phage selection. After a single round of phage display the percentage of positive clones sorted by yeast display is very low (0.1-1%), consequently the whole upper right quadrant is sorted in order to capture all potential antibodies. On subsequent sorts, the population becomes enriched, forming a diagonal population (FIG. 1) that broadly correlates antibody display with the amount of bound antigen. High affinity antibodies are selected by using diagonal sort gates on the upper border of the population. While in general high affinity antibodies are desirable, the exclusion of low affinity antibodies will lead to a loss of diversity. This may be detrimental if polyclonals with a greater number of antibodies recognizing many different epitopes are more effective as research reagents than polyclonals with fewer antibodies of higher affinity. This issue will be examined by generating polyclonals selected for different affinities using different sort gates and antigen concentrations to a number of different targets. These will be sequenced to assess diversity, and will be carried through the validation and characterization pipeline and compared, to determine the protocol that provides antibodies with the greatest specificity. In addition to positive selection for target binding, the possibility of introducing negative selection to eliminate polyreactive clones will also be examiner. These are usually due to truncated antibodies containing only a VH or VL domain, in which the hydrophobic interface binds non-specifically to other proteins. By including E. coli extracts, or a mixture of albumin, gelatin and milk, labeled with an orthologous dye (e.g. Alexa 647), it will be possible to sort the positive GFP labeled yeast, while simultaneously excluding those labeled with the non-specific proteins. Uhlén has described cases of monospecific PrEST antibodies that show specific cross-reactivity (Nilsson et al., Proteomics 5, 4327, 2005). We will use these PrESTs as a model to determine whether it is possible to also eliminate specific cross-reactivity during yeast display selection, once the cross-reactive species have been identified. If this is successful, we will be able to continually improve our renewable polyclonals by eliminating specifically cross-reactive species as they are identified during the characterization cycle.

Development of scFv-Fc Fusion Secretion Vector and Cloning of Yeast Selection Outputs

The most commonly used reagent antibodies are rabbit polyclonals and murine monoclonals. Rabbits have a single IgG Fc region (Martens et al., J Immunol 133, 1022, 1984), while mice have four: IgG1, IgG2a, IgG2b and IgG3. These all bind protein A and G (Akerstrom et al., J Immunol 135, 2589, 1985). New genes for each Fc region will be synthesized, with an N297Q mutation to eliminate the glycosylation site in CH2 (Tao and Morrison, J Immunol 143, 2595, 1989), and yeast codons to increase expression levels. Each Fc region will be fused to a high affinity anti-fluorescein scFv (4 m5.3; Boder et al., Proc Natl Acad Sci USA, 97 10701, 2000) to create the scFv-Fc format. The vector will be further modified to include high efficiency secretory leaders (Rakestraw et al., Biotechnol Bioeng 103, 1192, 2009) that have been evolved for improved antibody secretion. In initial experiments, the 4 m5.3 fused to the five different Fcs will be compared for secretion levels by ELISA recognition of fluorescein. This will be compared to results obtained using the cell surface secretion assay (CSSA) (Rakestraw et al., Biotechnol Prog 22, 1200, 2006; Manz et al., Proc Natl Acad Sci USA 92, 1921, 1995), in which secreted antibody is captured by fluorescein immobilized on the yeast cell surface and flow cytometry used to assess the amount of bound, and consequently secreted, antibody. Fc domains showing good expression and binding will be used for further improvement by directed evolution and DNA shuffling. CSSA will be used to select those mutated Fc domains imparting improved secretion to the S. cerevisiae clones that host them, using binding to both secondary antibodies and protein A/G as superimposed sort gates to ensure mutations do not eliminate reactivity to either. Improved Fc regions will be tested with additional antibodies. These will include previously selected high and low expressing scFvs as well as defined polyclonals, to assess the effects on secretion and recognition. Expression levels will be assessed, with a goal to consistently obtain at least 100 mg/L. Protocols will be optimized for expression in deep well microtiter plates.

Once a polyclonal antibody population has been selected in the yeast display vector it will be recloned to allow expression of the scFv-Fc protein. Cloning will be carried out by PCR amplification using vector encoded primers containing homologous sequences at both ends, allowing direct cloning of the scFv into the scFv-Fc S. cerevisiae expression vector described above. The process can be fully automated using robotics infrastructure and performed in the 96-well format.

### Using Deep Sequencing to Assess Diversity

It has been shown (Ravn et al., Nucleic Acids Res 38:e193, 2010; Di Niro et al., Nucleic Acids Res 38:e110, 2010) that sequencing of thousands of clones is required to identify the most enriched clones in a selected polyclonal population; the random screening of 96 clones is insufficient. For this reason it is believed that deep sequencing will be important for the assessment and characterization of diversity using different selection protocols, as well as in the potential informatic distribution of the selected monospecific polyclonals. 454 pyrosequencing (Ahmadian et al., Clin Chim Acta 363, 83, 2006) (50,000 reads of 250-400 bp) was used to assess diversity in a pool of potential interaction partners (Di Niro et al., Nucleic Acids Res 38:e110, 2010), whereas Ravn et al. (Nucleic Acids Res 38:e193, 2010) used Illumina (10⁷ reads of 76 bp). While the read length obtained with both of these platforms is too short to fully characterize a pool of selected scFvs (length ˜800 bp), the recently released Pacific Biosciences PacBio RS sequencing platform, based on the real time sequencing of single molecules (Eid et al., Science 323, 133, 2009), is able to provide ≧50,000 reads of ≧1000 bp, making it an ideal platform for the sequence characterization of scFv selections. Furthermore, due in part to the fast run-times and minimal technician intervention, the material cost per run (˜$100) is far less than that for the other platforms (˜$4000).

Within the context of pipeline development, the PacBio RS will be used to sequence selection outputs, and analyze the sequence data to determine which combinations of yeast and phage display retain the greatest diversity. This strategy will also be used to assess diversity loss sustained during required cloning procedures (phage to yeast display; yeast display to scFv-Fc secretion) and polyclonal pool amplification. Depending on the observed diversity, indexed libraries (barcoded) may be created to allow running a number of different polyclonal pools in a single sequencing run. The routine sequencing of selected polyclonal monospecific populations will be implemented in order to identify the top twenty clones in each population. Accurate error models are being developed and rigorous quality control of the resulting sequences will be performed.

A bioinformatic pipeline will be developed to perform quality trimming, separate the polyclonal pools by barcode, and assemble the long PacBio RS sequences, followed by read mapping and contig verification using unassembled reads, scFv sequence verification, and rank order those found more than 10 times. This will allow us isolate the most abundant scFv genes by inverse PCR using primers specific for the HCDR3 region.

### Tier 2 & 3 Selections for the Isolation of Specific Monoclonals

Tier 2B selection, as illustrated in FIG. 3, is currently the standard protocol for the selection of monoclonal antibodies using the combined phage and yeast display approach. Prior to isolating 96 independent clones to be tested for binding activity, the sorting gates are adjusted according to the clone properties required—e.g. greater specificity or higher affinity. After 2-3 rounds of yeast display selection, 90-100% of screened monoclonal antibodies recognize the target. A more labor and cost intensive method to select monoclonals, similar to that previously described (Ravn et al., Nucleic Acids Res 38:e193, 2010; Di Niro et al., Nucleic Acids Res 38:e110, 2010), will be used to carry out deep sequencing on the selection output, rank the clones in order of frequency and design inverse primers corresponding to each HCDR3, the most variable portion of the scFv, and that providing most of the binding activity (Xu and Davis, Immunity 13, 37, 2000; Nissim et al., EMBO J 13, 692, 1994) (tier 2A). Inverse PCR (Hoskins et al., Nucleic Acids Res 33, e185, 2005) is a method to clone plasmids for which sequence information is available, in which abutting primers are extended outwards to amplify the whole plasmid, prior to ligation. These primers will be used to amplify all plasmids containing the specific HCDR3s, and may also amplify clones with the same HCDR3 that do not bind because they are combined with alternative light chains. This approach requires the synthesis of two primers per selected monoclonal, but has the advantage that the most abundant antibodies will be isolated, rather than ones selected by chance. PCRs can also be carried out directly on the secretory polyclonal antibody pool, avoiding the need for additional cloning. The affinities of antibodies obtained by tier 2A and 2B methods will be compared to determine whether 2A is worth the extra effort. Tier 3 selection is a standard procedure involving the creation of error prone PCR libraries followed by flow cytometric sorting for improved affinity or specificity. The HT cloning procedures described above will be modified to introduce error prone PCR into clonal amplification, in order to carry out tier 3 selections in HT.

### Exploration of Generic Methods to Improve Affinities During the Selection Process

The affinities of the antibodies selected from the library range from 1-1000 nM. Traditionally the affinities of antibodies selected from naïve libraries have been improved by 1) chain shuffling (Lou et al., Protein Eng Des Sel 23, 311, 2010; Blaise et al., Gene 342, 211, 2004; Marks et al., Biotechnology (NY) 10, 779, 1992), in which the heavy chain is kept constant and shuffled against a library of light chains; and 2) error prone libraries and further selection (Boder et al., Proc. Natl. Acad. Sci. U.S.A. 97, 10701, 2000; Thie et al, Methods Mol Biol 525, 309, 2009; Razai et al, J Mol Biol 351, 158, 2005; Hawkins et al, J Mol Biol 226, 889, 1992 Gram et al, Proc Natl Acad Sci USA 89, 3576, 1992), an approach that is most effective when using yeast display. In addition, the coupling of two scFvs recognizing different epitopes can result in an enormous increase in affinity, due to a “chelate” effect (Neri et al., J Mol Biol 246, 367, 1995; Wright and Deonarain, Mol Immunol 44, 2860, 2007; Thou, J Mol Biol 329, 1, 2003). Chain shuffling has been traditionally carried out by restriction digestion and cloning. However, the use of the cre/lox system to generate initial diversity also potentially provides a means by which a modified chain shuffling protocol can be introduced into the selection pipeline with little additional effort. Unlike traditional chain shuffling, this does not match one VH with many different VL's, but recombines all VH's with all VL's. Within the context of the proposed phage-yeast selection protocol, the use of lox mediated chain shuffling during both phage and yeast selections will be explored, either using full selection outputs, or naïve libraries spiked with selection outputs. Phage recombination will be carried out as previously described (Sblattero and Bradbury, Nat. Biotechnol. 18:75, 2000), while recombination protocols for yeast will be developed, using either mating (Lou et al., Protein Eng Des Sel 23, 311, 2010; Blaise et al., Gene 342, 211, 2004; Weaver-Feldhaus et al, FEBS Lett 564, 24, 2004), or exploiting the finding that multiple plasmids can co-exist in yeast (Scanlon et al, BMC Biotechnol 9, 95, 2009), just as was previously shown for bacteria (Velappan et al., Protein Eng Des Sel 20, 309, 2007).

All publications describing increases in affinity using two scFvs binding to different epitopes have been carried out using antibodies for which the recognized epitopes are known. However, given the broad diversity of scFvs binding to a target of interest following in vitro selection, it is likely that many specific scFv pairs will give similar chelate effects when combined. The proposed use of the scFv-Fc format for antibody expression provides a straightforward method to combine such pairs of scFvs: the Fc domain will provide dimerization and the hinge the flexibility to allow two different scFvs to bind to a single target at two different epitopes. The possibility of creating heterodimeric bispecific scFv-Fcs in yeast will be explored using the same methods proposed above: mating or multiple plasmid transformation, starting with pairs of scFvs known to give a strong chelate effect (Neri et al, J Mol Biol 246, 367, 1995). Success in either or both of these methods may reduce the need to carry out tier 3 selections.

### Laboratory Information Management System (LIMS) Development

A sophisticated LIMS has been developed to track and manage both wet lab and computational workflows. Sample preparation and library construction operations in the laboratory are tracked and managed with the LIMS, and this will be extended to tracking of target preparation, antibody selection pools and associated metadata. Data generated by the sequencing lab will be automatically acquired, processed and stored on large capacity shared storage. Automated computational analysis is already performed and tracked by the LIMS system, with proper notifications to relevant personnel. The LIMS system is a configuration of the Genologic Life Science Geneus product customized for the operations described herein. Specifically, the LIMS will enable rapid configuration and automation of bioinformatic workflows, will interface with laboratory instrumentation for sample prep and library construction and track sample progress. It will also acquire and store results from laboratory instrumentation (sequencers) and automatically initiate multi-step computational analyses, in this case filtering reads of low quality, truncating vector sequences, assembly of remaining reads, and tallying and ranking of abundant antibody sequence types specific for a given target.

LIMS will provide the capability to search and access data, results, and sample information and generate customized reports. The LIMS system has a genealogy-like feature that greatly simplifies the organization and querying of sample metadata and results. The initiation of computational analysis and the association of results with samples for tracking and reporting is another important feature of this LIMS, which is able to automatically acquire raw data from sequencing instrumentation and kickoff initial analysis with no human intervention. This will save significant time, and provide a direct history of the algorithms and parameters associated with each sample and project.

### Characterization and Use of Renewable Recombinant Polyclonals

Internal validation

The output of the selection pipeline described above will be a pool of polyclonal antibodies raised against each target. Internal validation will be carried out on each polyclonal prior to external characterization. These will comprise non-specific binding, diversity (as determined by sequencing), specificity and affinity. In general, non-specific binding comes in two flavors. The first is general polyreactivity, in which an antibody binds to many different antigens. This is characteristic of some natural antibodies, and has been proposed to be an important component of the immune response (Thou et al, J Autoimmun 29, 219, 2007). It is also common in isolated VH or VL domains, and is likely to occur when antibodies are partially unfolded. Affinity purification of standard polyclonals drastically reduces non-specific binding (Nilsson et al., Proteomics 5, 4327, 2005). Specific cross reactivity occurs when antibodies recognize targets sharing epitopes. It is usually sufficient to screen an antibody against a small panel of additional targets to identify general polyreactivity, while the elimination of specific reactivity will require screening against all other proteins, which is not yet feasible. Initial specificity will be assessed by a published multiplexed flow cytometry method (Ayriss et al., Methods Mol Biol 525, 241, 2009; Ayriss et al, J Proteome Res 6, 1072, 2007), using a 30plex bead set coupled to the specific recognized target, a panel of irrelevant targets, protein G and protein A, the latter to assess expression levels. This will be sufficient to identify general polyreactivity in the selected polyclonals. Depending upon the levels of polyreactivity, the value of negative rounds of yeast display selection will be assessed, carried out as described above, to reduce polyreactivity. Identified levels of non-specific binding obtained using the scFv-fusions will be compared to those when the scFv is displayed on yeast. If they are similar, it will be possible to reduce non-specific binding prior to scFv-Fc expression.

A number of studies have shown that the affinity measurement of monoclonal antibodies displayed on yeast gives results similar to those obtained by surface plasmon resonance (Chao et al., Nat Protoc 1, 755, 2006; Lou et al, Protein Eng Des Sel 23, 311, 2010; Razai et al, J Mol Biol 351, 158, 2005; Colby et al, Methods Enzymol 388, 348, 2004; Garcia-Rodriguez et al, Protein Eng Des Sel, 2010), providing a rapid method to assess monoclonal antibody affinity. The tier 1 antibodies will, however, be polyclonals, and attempts to determine the affinity of polyclonal antibodies has either involved the fractionation of polyclonal antibodies into different affinity bins (Voss et al, Mol Immunol 38, 35, 2001) or considered the polyclonal antibody as if it was a single species (Cachia et al, J Mol Recognit 17, 540, 2004; Sem et al, Arch Biochem Biophys 372, 62, 1999), with the calculated affinity representing the weighted mean of individual antibodies (Sem et al, Arch Biochem Biophys 372, 62, 1999). A recent molecular analysis (Poulsen et al, J Immunol 179, 3841, 2007) indicated that the human immune response to tetanus toxin involves ˜50 V gene rearrangements with affinities spanning 10⁶ orders of magnitude for the total population and 10-100 fold for any one clonal group. The polyclonal antibody pool will be considered a monoclonal species and affinity will be calculated by a progressive titration of target fluorescently labeled with split GFP. This method has the advantage that functional target concentrations can be rapidly calculated on the basis of their fluorescence. In addition, the affinity range will be determined by measuring the coefficient of variation of antigen binding at the different concentrations.

### External Characterization

Once antibodies have been validated and shown to be monospecific they will be expressed as scFv-Fc fusions in S. cerevisiae and characterized. The first polyclonals will be selected against PrEST targets shown to provide consistent reliable characterization data and will be taken through the full characterization pipeline (see proteinatlas.org and Uhlen et al., Nat Biotechnol 28, 1248, 2010; Uhlen and Hober, J Mol Recognit 22, 57, 2009; Hober and Uhlen, Curr Opin Biotechnol 19, 30, 2008; Berglund et al, Mol Cell Proteomics 7, 2019, 2008; Uhlen et al, Mol Cell Proteomics 4, 1920, 2005) comprising specificity (antigen/PrEsST array), target molecular weight (western blotting), tissue distribution (immunohistochemistry on normal and tumor tissues) and subcellular distribution (confocal immunofluorescence on three cell lines).

Two additional characterization pipelines will be developed: 1) In depth multiplexed flow cytometry specificity; and 2) immunoprecipitation and subcellular localization. It will not be possible to screen for non-specific binding to all human proteins, as it is unlikely that it will be possible to recombinantly express and purify each one. Consequently, the ability to screen each polyclonal antibody against a broad range of soluble PrESTs will provide a valuable measure of cross-reactive potential, even if not full comprehensive. To this end, as soluble PrESTs become available, they will be coupled to streptavidin coated multiplexed beads, using the in vivo biotinylation tag. The eventual goal is to have a multiplex antigen array comprising 5,000 different proteins against which each polyclonal can be screened in a few minutes.

In addition to coupling targets to beads, the selected polyclonals will be used within an antibody array context to determine 1) the molecular weight of targets in their natural states; 2) the subcellular localization of recognized targets and eventually 3) identify the interaction partners of the proteins within the cell. This will be an extension of work already carried out (Wu et al., Mol Cell Proteomics 8, 245, 2009). The polyclonal antibodies are first bound to color-coded beads. A mixture of beads is prepared where each color code corresponds to a different polyclonal antibody. The bead mixture is added to microtiter wells containing biotinylated cellular proteins that have been fractionated according to subcellular location and size. The cumulated data output provides size distribution profiles for the proteins identified by each polyclonal antibody. Unlike western blotting, this provides native recognition. It has been found that antibodies often bind targets that are much larger than the monomeric form of the intended target, in many cases corresponding to multi-protein complexes. The identity of the components of these complexes can be determined by eluting proteins binding to antibodies and applying them to either a second antibody bead array, or to a slide based antibody microarray.

An additional component of the antibody characterization pipeline will be the development of direct and on demand antibody arrays, exploiting the availability of both antibodies and antibody genes provided by the selection pipeline. These will be complementary to the multiplexed bead arrays, and will be particularly useful for the identification of components of immunoprecipitated complexes. For the direct arrays, the use of scFv-Fc fusions will be explored arrayed directly, or onto slides precoated with protein A or G, both of which will bind to the Fc domain of the polyclonal antibodies, and which have been shown to improve antibody orientation (Matson et al, Methods Mol Biol 382, 273, 2007; Choi et al, Ultramicroscopy 108, 1396, 2008; Clarizia et al, Anal Bioanal Chem 393, 1531, 2009). The results obtained with these arrays will be compared to those obtained by on demand arrays, in which yeast polyclonal antibody genes will be used as a source of antibody diversity. The scFv fragments will be amplified using overlapping oligonucleotides providing overlaps in order to assemble final PCR fragments comprising T7 promoter-scFv-VirD2-SV5 tag-6H is. PCR fragments will be spotted on nitrocellulose slides with the T-border oligo. After DNA cross-linking, arrays can be stored. When required, optimized in vitro transcription translation reactions will be performed to obtain a fully functional array. All arrays will be evaluated to assess the level of antibody expression using labeled SV5 (an antibody that recognizes a peptide tag on the scFv), the expression cv for different PCR amplifications (i.e. assessing potential bias during amplification); the stability over time and under different storage condition. Finally the array will be challenged first with purified targets and non-specific proteins, labeled with biotin, and subsequently protein extracts (as carried out for the multiplex beads array). Bound proteins will be labeled with CY3 or CY5, the signal measured using the ScanArray Gx®, PerkinElmer and analyzed with ScanArray Expression Software.

### Example 5

### Use of Phage and Yeast Display to Select Hundreds of Monoclonal Antibodies

This example describes a method that combines phage display and yeast display to select antibodies against any target antigen. In this example, tuberculosis (TB) antigen 85 (Ag85) is used as the target antigen.

### Materials & Methods

### Strains

DH5αF′: F′/endA1 hsdR17(rK-mK+) supE44 thi-1 recA1 gyrA (Nair) relA1 D(lacZYAargF) U169(m80lacZDM15) was used for phage display and sequencing analysis. S. cerevisiae EBY100 (GAL1-AGAE:URA3 ura3-52 trpl leu21 his3200 pep4::HIS2 prb11.6R can1GAL) was used for yeast display (Boder and Wittrup, Biotechnol Prog 14(1):55-62, 1998).

### Antigen Preparation

The purified Ag85 complex and Ag85A, Ag85B and Ag85C components were obtained from BEI Resources, the purity of which was checked by SDS-PAGE. Purified Ag85 proteins were biotinylated using the Lightning-Link® Biotin kit (Innova Bioscience) following manufacturer's instructions.

Phage Display Selection of scFv Antibodies

A previously described naïve phage antibody library (Sblattero and Bradbury, Nat Biotechnol 18:75-80, 2000) was used to select Ag85 antibodies using an automated Kingfisher magnetic bead system (Thermo Lab Systems), allowing selection to be carried out in solution as previously described (Marks and Bradbury, Methods Mol Biol 248:161-176, 2004). 0.5 μg of biotinylated Ag85 were used in the first round and 0.05 μg in the second. Capture of phage binding to biotinylated Ag85 was carried out using 2×10⁷ streptavidin magnetic beads (Dynabeads M-280).

Yeast Display of Anti-Ag85 scFvs

After two phage selection rounds DNA encoding the selected scFv antibodies was prepared using the QIAprep™ spin miniprep kit (Qiagen) and used as a PCR template for amplification with pDan5topDNL6-5′ (TCTGTGTGGTGGTGCGGCGCGCATGCC; SEQ ID NO: 1) and pDan5topDNL6-3′ (ATCCAGGCCCAGCAGT-GGGTTTGGGATTGGTTTGCC; SEQ ID NO: 2). The PCR product is compatible with the yeast display vector pDNL6 (a pPNL6 (Feldhaus et al., Nat Biotechnol 21:163-170 2003) derivative compatible with pDAN5 (Sblattero and Bradbury, Nat Biotechnol 18:75-80, 2000)), allowing cloning by gap repair (Boder and Wittrup, Nat Biotechnol 15(6):553-557, 1997).

The yeast mini-libraries were further enriched by one or two rounds of sorting using flow cytometry using published methods (Boder and Wittrup, Nat Biotechnol 15(6):553-557, 1997). Briefly, 2×10⁶ yeast cells were washed and resuspended in 100 μl of wash buffer containing 100 nM of biotinylated Ag85. Cells were labeled with streptavidin-AlexaFluor633 to detect binding of biotinylated Ag85 and 1 μg of anti-SV5-PE to assess scFv display levels. Flow cytometry was performed using the FACSAria (Becton Dickinson), with yeast showing Ag85 and SV5 binding sorted. Collected cells were grown overnight at 30° C. and induced for the next round of sorting. The sequences of sorted antibodies were determined on plasmid DNA prepared in DH5αF′ after transformation and clones were grouped based on the analysis of the CDRH3 region.

One hundred eleven (111) different scFvs were obtained. Forty-eight of these were induced and tested for specific binding by flow cytometry (LSRII, Becton Dickinson) using biotinylated Ag85; myoglobin and streptavidin-Alexa-Fluor633 were used as negative controls. The 48 clones were also tested for their binding activity for the different subunits (A, B and C) forming the Ag85 complex. The experiment was done in duplicate and standard deviation was calculated. BDiva software was used for flow cytometric analysis. The affinities of fourteen of the 48 clones were calculated directly on the yeast surface, which shows excellent agreement with other methods (Gai and Wittrup, Curr Opin Struct Biol 17(4):467-473, 2007). Approximately 10⁶ induced yeast cells were incubated with a dilution series of Ag85 complex (0-200 nM) detected as described above. The LSRII was used for flow cytometric analysis and BDiva software used for analysis. Quantitative equilibrium constants were calculated by plotting the streptavidin-AlexaFluor-633 normalized (APC antigen binding signal) mean fluorescence intensity against antigen concentration and fitting the curve using nonlinear regression analysis.

Secretion and Purification of Soluble scFv-Fc Fusions and their Use in ELISA

scFv were subcloned into a modified pPNL9 vector (Siegel et al., J Immunol Methods 286(1-2):141-153, 2004) which expresses scFv-Fc fusions with rabbit Fc, with an SV5 tag at the C-terminus, allowing detection with anti-SV5 or anti-rabbit secondary reagents. After transformation into YVH10 (Orr-Weaver and Szostak, Proc Natl Acad Sci USA 80(14):4417-4421, 1983), culture supernatant was used directly as a reagent after growth for 24-48 hours at 20° C. Binding of the scFv-Fc fusions was assessed by ELISA, using antigens, Ag85, ESAT6 (a different soluble protein produced by M. tuberculosis) and myoglobin, directly coupled to Nunc immunosorb microtiter plates, and 1/5 diluted yeast expression culture. Binding was detected with anti-SV5 HRP (horseradish peroxidase) or anti-rabbit-HRP. The produced scFv-Fc fusions were also tested for their stability over time. The concentration of recombinant antibodies was calculated at 1 mg/L and 100 μl of this solution, after different periods of storage, were used in ELISA assays as described above. A commercial rabbit monoclonal antibody (AbCam) at the same concentration was used as a standard, with its absorbance used to normalize the yeast expressed scFv-Fc activity. All experiments were carried out in triplicate and the standard deviation was calculated.

### Sandwich Assay

To determine detection limits, a sandwich assay was performed using yeast displayed antibodies and scFv-Fc fusions expressed from yeast. Eight of the anti-Ag85 scFv tested in ELISA were cloned into the yeast display vector pDNL6 and transformed into S. cerevisiae EBY100. Single clones were induced and resuspended in serial Ag85 dilutions (ranging from 5-100 nM). After incubation and washing, the yeast cells, with bound Ag85 attached, were resuspended in culture supernatants containing secreted scFv-Fc fusions. After incubation and washing, cells were resuspended in anti-rabbit-Alexa633 (Invitrogen, Carlsbad, Calif.) to detect soluble scFv-Fc bound to Ag85. scFv-Fc fusions recognizing bound Ag85 at a site different to the yeast displayed scFv will be able to bind the Ag85 and confer a positive signal to the yeast. After washing and resuspension in PBS, fluorescence data were collected using the FACSAria and analyzed using DIVA software. The yeast population was gated and the mean fluorescent values for allophycocyanin (APC) (corresponding to the binding of scFv-Fc to antigen immobilized by the yeast displayed antibodies) were recorded using appropriate excitation lasers and emission filters. The same assay was used to test the detection of Ag85 in human serum, except that antigen was serially diluted in 1:50 serum diluted in PBS.

### Results

Selection of Ag85-Binding Clones from a Naïve Human Library

The strategy to select scFvs against Ag85 using phage and yeast display is described below. Phage display is used to preselect the vast diversity of the naïve phage antibody library to a diversity compatible with subsequent analysis and selection by yeast display. After two rounds of phage selection, diversity was reduced from ˜10¹¹ different scFv (Sblattero and Bradbury, Nat Biotechnol 18:75-80, 2000) to ˜70,000 colony forming units. This was cloned into the yeast display vector by gap repair (Boder and Wittrup, Nat Biotechnol 15(6):553-557, 1997) with a greater than ten-fold coverage of the phage scFv output, ensuring maintenance of diversity. 22.2% of yeast displayed a functional scFv, of which 10% (2.1% of total population) bound Ag85, and less than 0.5% (0.1% of total) showed non-specific binding to streptavidin.

### Yeast Display Mini-Library Sorting

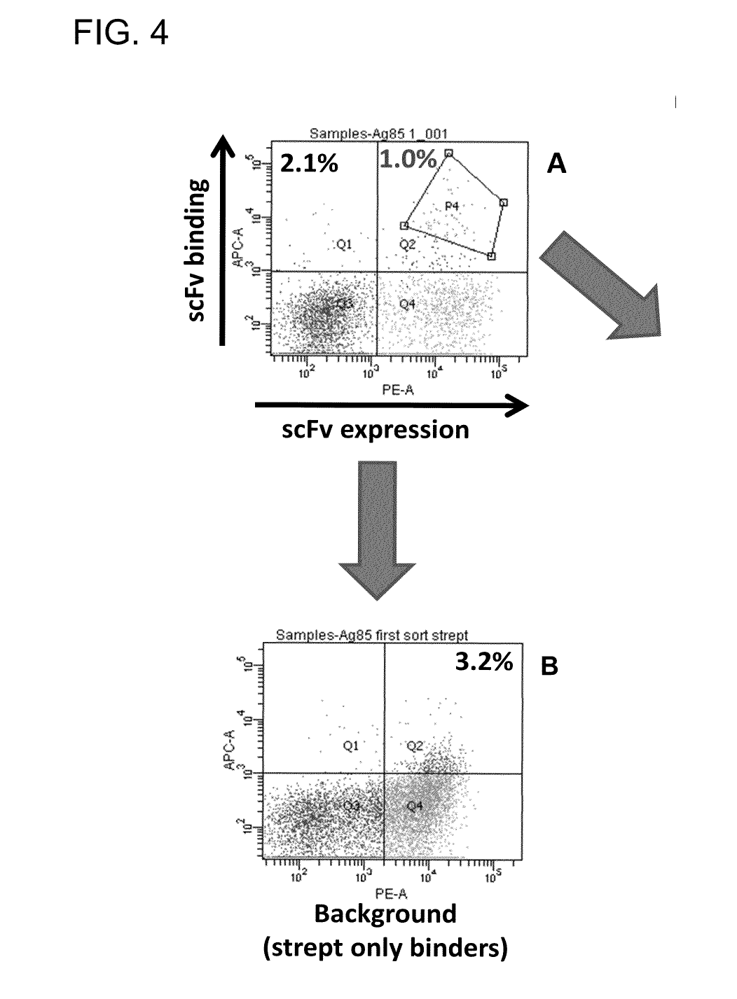

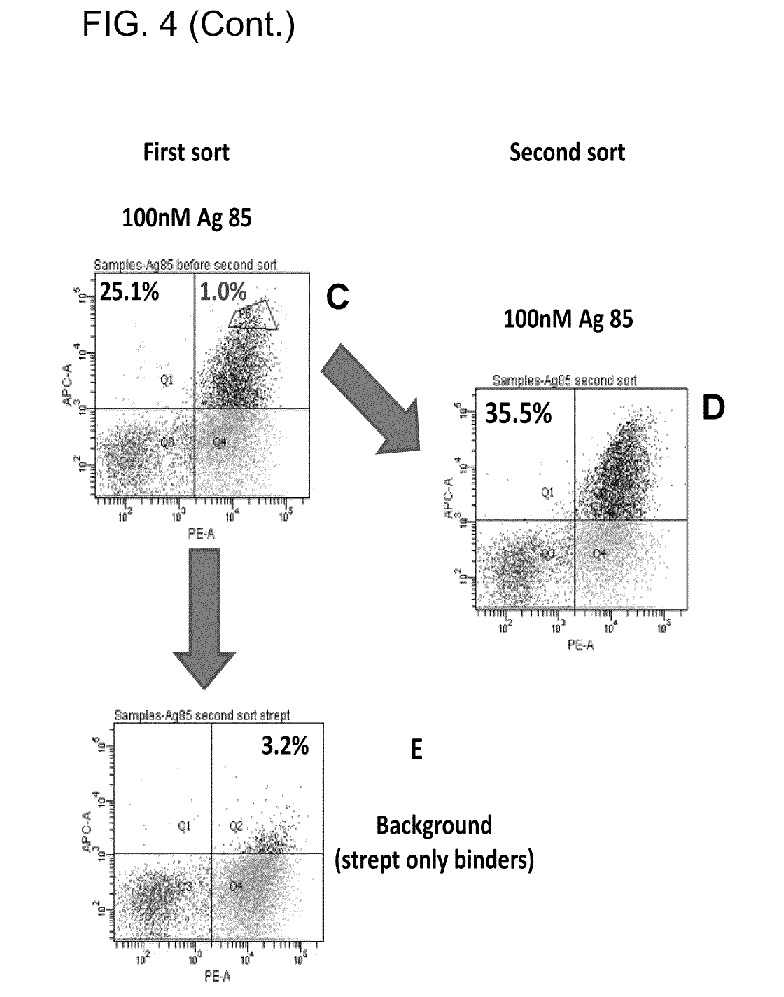

Yeast displaying Ag85 binding scFvs were sorted by incubating 2×10⁶ induced yeast mini-library cells with 100 nM of biotinylated Ag85, streptavidin-AlexaFluor633 and phycoerythrin labeled anti-SV5 to measure scFv display levels. The top 1% of Ag85 binding yeast was sorted, using the P4 gate in FIG. 4A, grown in selective media and regrown in induction media prior to reanalysis and repeat sorting (FIG. 4B). After one sort, the percentage of yeast displaying scFvs recognizing Ag85 reached 25.1%. After sorting the top 1% of this population (sort gate P5 in FIG. 4B) the percentage of Ag85 binding cells increased to 35.5% (FIG. 4C), while the percentage of streptavidin binding clones remained constant at 3.2% for both (FIGS. 4D-4E).

### Monoclonal Antibodies Displayed on Yeast

Sequencing of 192 random clones after each of the two rounds of flow sorting (96 each) revealed a total of 111 different scFvs: 96 unique clones to either one or two rounds of yeast sorting and 15 common to both. Forty-eight of these, including the 15 shared ones, and an additional 33 found more than once in the sequencing, were tested for binding to Ag85 and two non-related proteins (ubiquitin and myoglobin). The mean fluorescent signal for each of these was clearly specific for Ag85, with very low recognition of the controls, indicating that at least 48 and probably more than 100 different specific antibodies against Ag85 were isolated by this combined phage/yeast display approach.

### Antibody Response to Ag85 Complex and its Components

All the enrichment steps (phage selections and yeast display sorting) were performed using the Ag85 complex as target. However, as described above, Ag85 is composed of three subunits, Ag85A, B, C, which have 71-77% homology. For this reason, it was decided that yeast display would be used to test the antibodies for their ability to recognize the individual Ag85 components. Binding activity to the complete Ag85 complex was used to normalize the recognition of the 48 different scFvs for the three different components. None of the clones analyzed showed a strictly subunit specific binding activity, with a majority recognizing each of the subunits equally well. However, there were interesting patterns in which one or two of the subunits were clearly better recognized. For example, two clones (c2 and b11) showed a preference for Ag85A and C over Ag85B, while b7 recognized Ag85 B and C, and showed no binding to Ag85A. Other clones (e.g. a7, a12, b3, c7 c12 and d8) showed a preference for Ag85B. No clones preferentially recognizing Ag85A or Ag85C were isolated. The clones indicated with arrows were taken forwards for affinity analysis.

### Affinity

The affinities of the fourteen most interesting clones (in terms of binding profile during the screening and for their activity against the different Ag85 components) were calculated by incubating each yeast-displayed scFv with varying quantities of Ag85 complex (Gai and Wittrup, Curr Opin Struct Biol 17(4):467-473, 2007). The binding constants for the scFv clones ranged from 22.1 nM to 432 nM with an average affinity of 126.1 nM, very similar to the 100 nM concentration of Ag85 used for yeast-based selection. The affinities of the antibodies obtained after one round of sorting had a broader range (39-432 nM, mean 180 nM) than those after two rounds of sorting (59.9-160 nM, mean 120.4 nM) or those found in common (22.1-110 nM, mean 81.8 nM) (Table 1).

TABLE 1
Affinities of 14 antibodies determined directly on the surface of yeast
Antibodies are identified by whether they were isolated after
one yeast sort (1 sort), two yeast sorts (2 sort), or were found
in common to both yeast sorts (com scFv).

| | Kd scFv anti-Ag85 | group |
|---|---|---|
| A2 | 39 | 1 sort |
| A3 | 99.5 | 1 sort |
| A4 | 151 | 1 sort |
| A7 | 432 | 1 sort |
| av 1 sort | 180.375 | |
| B10 | 98 | com scFv |
| B12 | 110 | com scFv |
| C2 | 97 | com scFv |
| E6 | 22.1 | com scFv |
| av com scFv | 81.775 | |
| C9 | 160 | 2 sort |
| C10 | 59.9 | 2 sort |
| C11 | 120 | 2 sort |
| D3 | 84.5 | 2 sort |
| D8 | 202 | 2 sort |
| F1 | 96 | 2 sort |
| av 2 sort | 120.4 | |
| total average | 126.1 | |

### ELISA

Having selected large numbers of different scFvs with different affinities and recognition specificities for the different components of the main antigens, 14 scFvs were re-cloned into a yeast expression vector as scFv-Fc fusions (Powers et al., J Immunol Methods 251(1-2):123-135, 2001). These are constructs in which the scFv is directly fused to the N-terminus of the hinge region of an immunoglobulin Fc region, and have the advantage that they are far more stable than scFvs alone, and preserve the binding properties of the original scFv. Furthermore, by using an Fc region derived from rabbit, it is possible to use standard immunochemical secondary reagents. This allowed for testing of the ability of these selected scFvs to specifically recognize Ag85 directly adsorbed to plastic in an ELISA format using standard anti-rabbit antibodies as secondary reagents. The results showed that 11 of the 14 antibodies bind to Ag85 when adsorbed to plastic, without recognizing ESAT6 and myoglobin, the negative controls. Given that all 14 recognized Ag85 by flow cytometry, it is likely that the three negative antibodies (A3, A7 and D3) recognized conformational epitopes that may have been lost by adsorption to plastic, as previously described for other proteins (Butler et al., Immunol Invest 26(1-2):39-54, 1997).

### Sandwich Detection of Ag85

On the basis of these results, the top eight clones were further tested in an 8×8 sandwich assay, in which yeast displaying all eight selected antibodies were used as Ag85 capture reagents, and tested with all eight scFv-Fc fusions as detection reagents, with detection assessed using fluorescent anti-rabbit secondary antibodies. Prior to carrying out the sandwich assays, the yeast displaying each of the eight antibodies were shown to bind fluorescently labeled Ag85, indicating their functionality as potential capture agents.

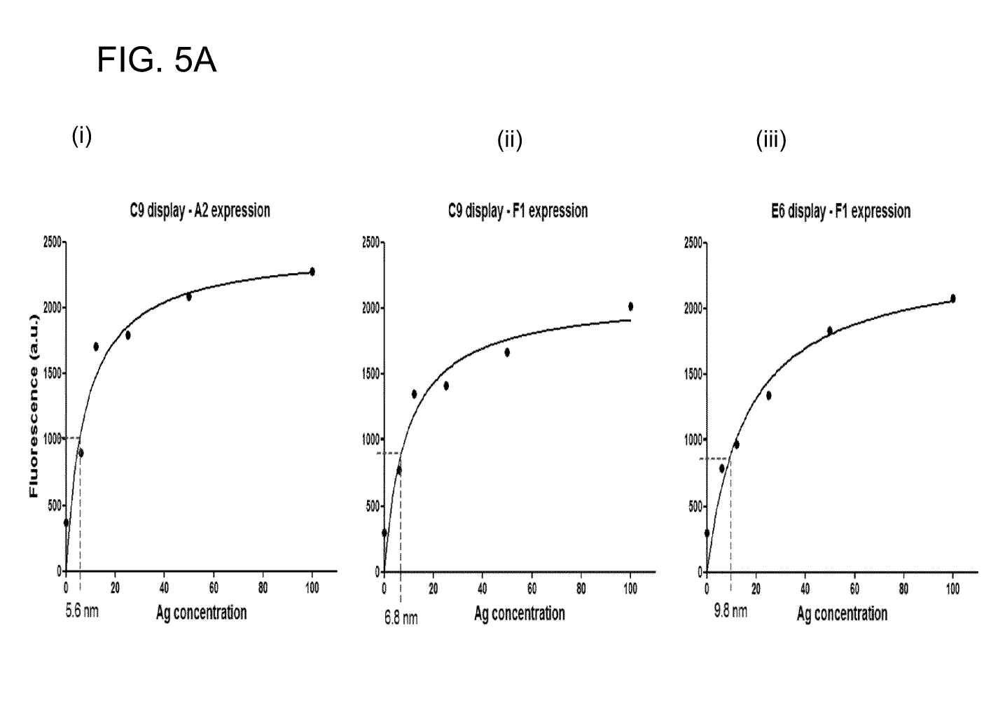

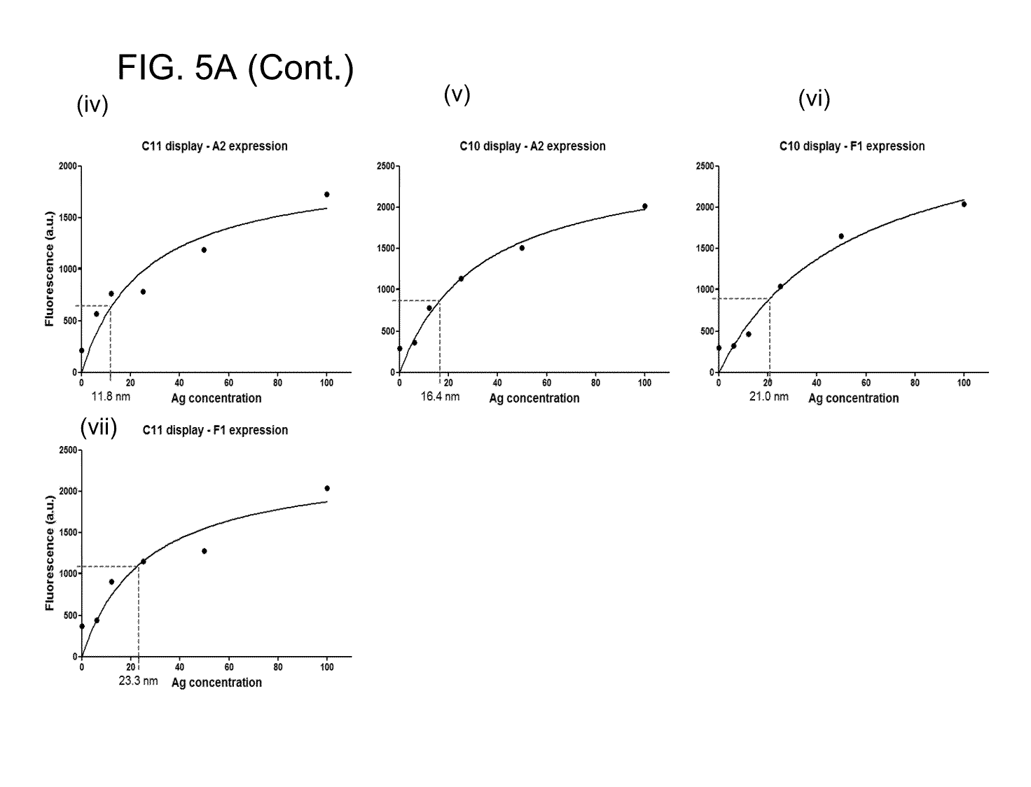

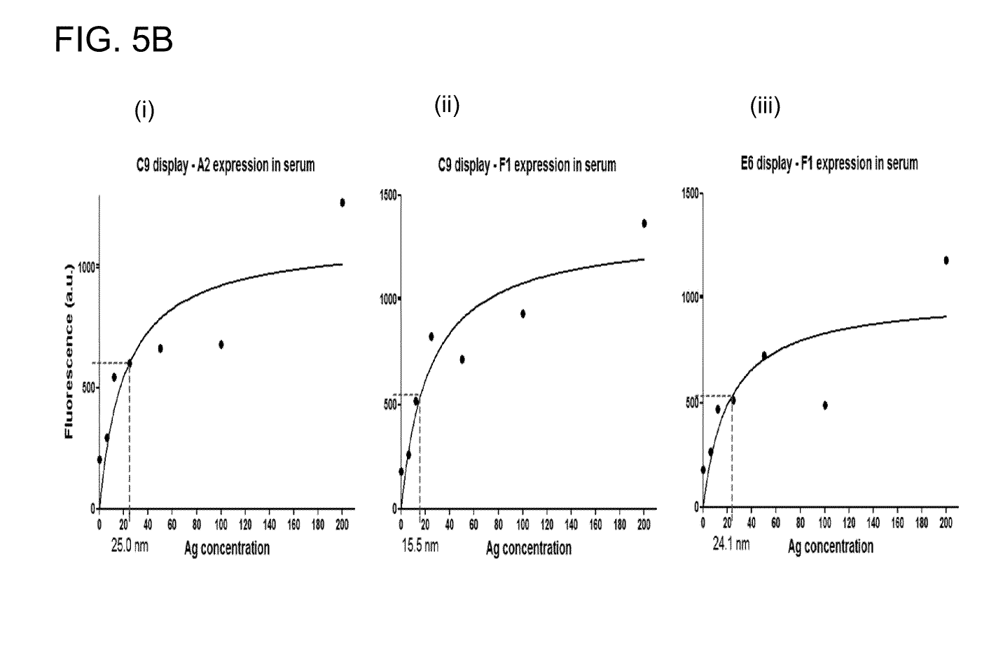

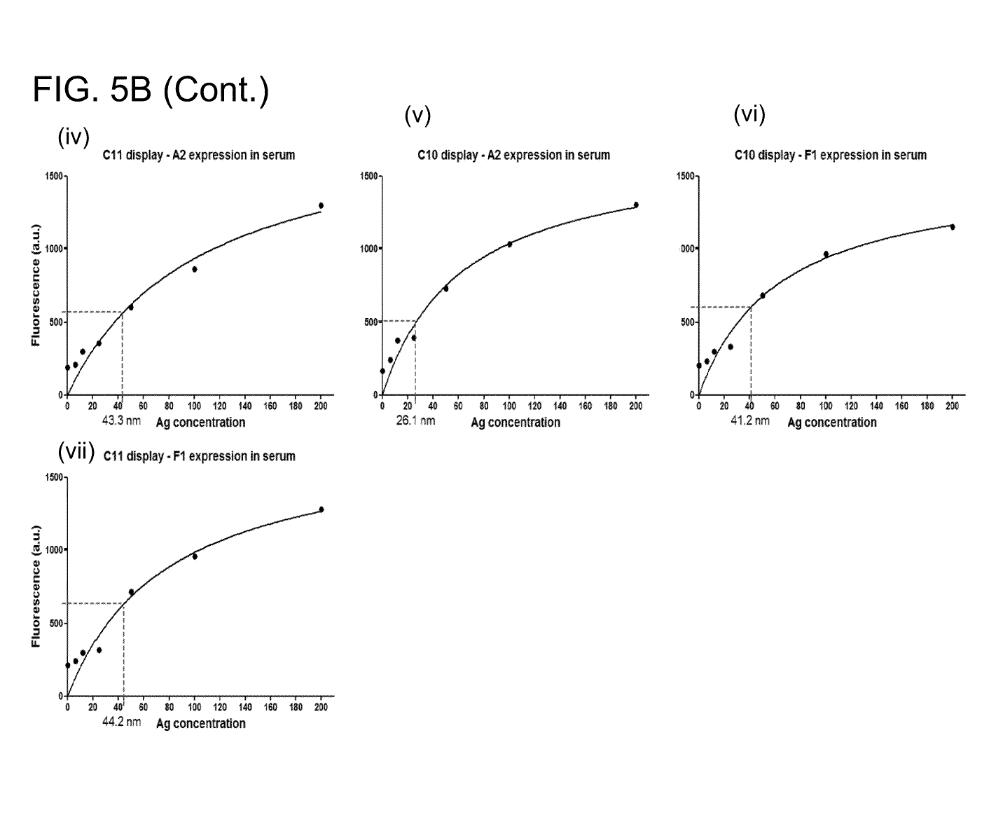

The results show that approximately a third of antibody pairs (21 of 64) showed a clear shoulder (e.g. C10 display; A2 scFv-Fc) indicating a positive result in the sandwich assay. Three of the antibodies (C2, C9 and C11) gave positive results when used simultaneously as both capture and detection reagent, reflecting the fact that Ag85 is a trimer and that these antibodies (as well as most of the others) are able to recognize more than one subunit. Antibodies A2, B12 and F1 did not appear to give positive signals for any of the scFv-Fc fusions when used as capture agents, even though they were functional when used in the reverse formats (e.g. compare C10 display and A2 scFv-Fc with A2 display and C10 scFv-Fc). The remaining five displayed antibodies (C2, C9, C10, C11 and E6) were tested with all the scFv-Fc fusions to establish detection limits. The results of the best pairs are illustrated in FIG. 5A, showing detection down to 6.1 nM in the absence, and 22.7 nM in the presence, of serum for the best pair (C9 display and A2 scFv-Fc expression) (FIG. 5B).

Stability of the scFv-Fc Fusion

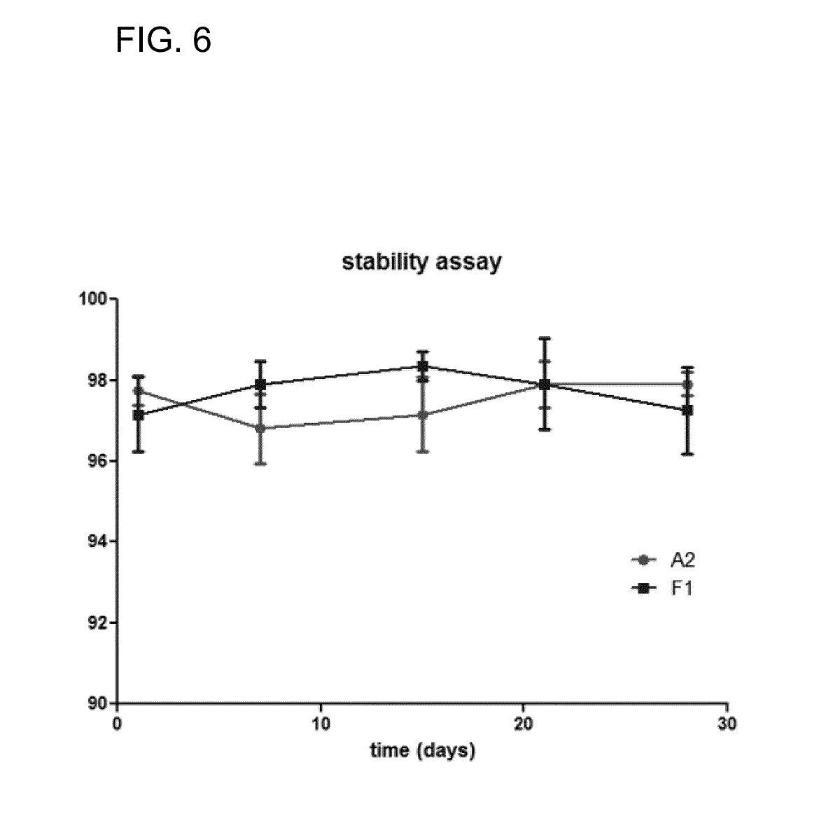

One of the main problems with the commercially available antibodies when used for Ag85 detection is their tendency to be unstable, resulting in either a loss of binding activity, or increased non-specific binding, even when stored as recommended. In order to determine whether in vitro selected scFv-Fc fusions are more stable, the stability of two of the unpurified antibodies was tested for a period of four weeks at 4° C. The binding activity of the antibodies was detected in ELISA (FIG. 6), and normalized with respect to binding on the first day. As shown in FIG. 6, there is no significant variation in the signal during this period, indicating the stability of these constructs without the need for a purification step.

### Discussion

The direct detection of Ag85 in patient serum has been proposed as an effective diagnostic for TB (Kashyap, et al., BMC Infect Dis 7:74, 2007; Landowski et al., J Clin Microbiol 39(7):2418-2424, 2002). This requires stable specific high affinity antibodies, which to date have been either polyclonal (Landowski et al., J Clin Microbiol 39(7):2418-2424, 2002; Bera et al., Indian J Pediatr 73(8):675-679, 2006) or monoclonal (Kashyap, et al., BMC Infect Dis 7:74, 2007; Landowski et al., J Clin Microbiol 39(7):2418-2424, 2002; Bentley-Hibbert et al., Infect Immun 67(2):581-588, 1999), obtained by immunization. More recently, methods to generate antibodies using in vitro methods, such as phage and yeast display, have been developed. These have significant advantages over traditional immunization, including the speed with which antibodies can be selected and the ability to screen many more clones for highly specific properties. More significantly, once selected, it is relatively straightforward to improve many antibody properties such as affinity (Boder et al., Proc Natl Acad Sci USA 97(20):10701-10705, 2000; Yang et al., J Mol Biol 254(3):392-403, 1995), specificity, (Garcia-Rodriguez et al., Nat Biotechnol 25(1):107-116, 2007; Bostrom et al., Science 323(5921):1610-1614, 2009) and expression levels (Shusta et al., J Mol Biol 292(5):949-956, 1999; Boder and Wittrup, Methods Enzymol 328:430-444, 2000). The present disclosure describes combining the two most powerful in vitro antibody selection platforms (phage and yeast display) to select a large number of antibodies against a target antigen, as exemplified by Ag85. This panel comprises a far greater antibody variety than usually obtained when carrying out phage display alone, significantly increasing the number of available anti-Ag85 antibodies. It is believed that this is due to the more efficient harvesting of the available antibody diversity. Depending upon the efforts expended in creating them, naïve antibody libraries have enormous diversity (>10⁹ different clones) (Feldhaus et al., Nat Biotechnol 21:163-170 2003; Sblattero and Bradbury, Nat Biotechnol 18:75-80, 2000; Sheets et al., Proc Natl Acad Sci USA 95(11):6157-6162, 1998; Vaughan et al., Nat Biotechnol 14(3):309-314, 1996). Theoretical (Perelson and Oster, J Theor Biol 81(4):645-70, 1979) and practical (Marks et al., J Mol Biol 222(3):581-597, 1991; Griffiths et al., EMBO J 13(14):3245-3260, 1994) studies have shown that, depending upon the desired affinity, a library size of ˜10⁷ clones should yield approximately one specific antibody, and that the number of selected antibodies should increase proportionally with library size. However, this has not been the case with phage display, where, unless especially heroic efforts are made (Edwards et al., J Mol Biol 334(1):103-118, 2003), only 5-50 different specific antibodies are usually selected from large libraries. This is likely due to additional biases occurring during phage display selection (de Bruin et al., Nat Biotechnol 17(4):397-399, 1999) that do not appear to occur in yeast display (Feldhaus et al., Nat Biotechnol 21:163-170 2003), probably because expression control is tighter.

This combination of technologies exploits the advantages of each of the two platforms. After carrying out one to two rounds of selection in phage, the diversity is reduced from 10⁹⁻¹¹ to 10⁵⁻⁶. This is compatible with straightforward cloning into yeast, and exploits the tremendous flexibility and sensitivity of the use of flow cytometry in subsequent antibody selection. Whereas traditionally up to 384 different clones are tested by ELISA after phage display, when phage and yeast display are combined as described here, every phage selected clone is tested individually: analogous to carrying out up to one million ELISAs, with the ability to sort out those that are positive in real time. This represents a significant advantage over using either phage or yeast display alone. In the former, the full selected diversity cannot be tested, while in the latter, it is already challenging to screen the naïve 10⁹ clone yeast antibody library that is available (Feldhaus et al., Nat Biotechnol 21:163-170 2003), and significantly more so to access even larger initial diversities. By combining the display platforms as described here, all potential positives in a phage library can be screened.

Using this approach, 111 different specific positive antibodies were identified out of only 192 sequenced clones. It is expected that the application of deep sequencing to the selection (Ravn et al., Nucleic Acids Res 38(21):e193, 2010; Di Niro et al., Nucleic Acids Res 38(9):e110, 2010; Glanville et al., Proc Natl Acad Sci USA 106(48):20216-20221, 2009) will identify significantly more positive clones. Although many different antibodies recognizing Ag85 were identified, none were identified with absolute specificity for one of the subunits over the others. In part, this is likely to be due to the way the selection was carried out: using a trimer of similar subunits the dominant epitopes will be those found in all three subunits. Future attempts to select subunit specific antibodies will involve selection against one subunit in an excess of the others.

The data presented here focus on Ag85, a soluble antigen. However, this technology is not restricted to soluble protein antigens, but can also be applied to the isolation of antibodies against membrane proteins. Both phage (Marks et al., Biotechnology 11(10):1145-1149, 1993) and yeast (Richman et al., Protein Eng Des Sel 19(6):255-264, 2006; Wang et al., Nat Methods 4(2):143-145, 2007) display have been used to isolate antibodies in whole-cell panning experiments, and more recently specific protocols have been developed that allow the selection of antibodies against either random, or specific, membrane proteins (Cho and Shusta, Protein Eng Des Sel 23(7):567-577, 2010). Combining these well described methods with the approach described here will allow the straightforward selection of antibodies against many different transmembrane proteins.

From these original 111 identified positive antibodies, a series of rapid screening assays were carried out, leading to 7 antibody pairs able to detect Ag85 down to 6.1 nM in the absence of serum and 22.7 nM in its presence. One attractive feature of this combined technology is the large number of different preliminary assays that can be carried out without the need to express or purify the antibodies. In particular, yeast displayed antibodies can be considered to be bead immobilized antibodies that only need to be grown up and induced in order to determine their affinity, specificity and epitope binding.

The direct detection of Ag85 components in serum is regarded as one of the most promising direct antigen detection potential diagnostic tests for TB (Kashyap, et al., BMC Infect Dis 7:74, 2007; Landowski et al., J Clin Microbiol 39(7):2418-2424, 2002), particularly because of the very high level of secretion of this target (Fukui et al., Biken J 8(4):189-199, 1965). This work describes a broad new diverse panel of anti-Ag85 (human) monoclonal antibodies, with great potential for the study and diagnosis of TB.

### Example 6

### Recombinant Renewable Polyclonal Antibodies

This example describes the finding that potent recombinant renewable polyclonal antibodies, in which essentially all antibodies are directed to the target of interest, can be derived from naïve phage antibody libraries, in combination with yeast display.

### Materials and Methods

### Antigens

Antigens were provided by Structural Genomic Consortium (Canada). Quality assessment was performed by mass spectrometry, SDS-PAGE and Western Blot, to assess biotinylation.

### Phage-Display Selection

Selections of scFv antibody fragments by phage display from the naïve library (Sblattero and Bradbury, Nat Biotechnol, 18(1):75-80, 2000) was performed as described in Example 5. Briefly, selections were carried out in parallel in solution (Marks and Bradbury, Methods Mol Biol, 248:161-76, 2004), using the automated Kingfisher magnetic bead system (Thermo Lab Systems): 0.5 μg of biotinylated antigen were used in each selection cycle. The biotinylated antigens were incubated with the naïve library and the bound phages were captured using 2×10⁷ streptavidin magnetic beads (Dynabeads M-280).

Yeast Display and Sorting of scFvs

After two rounds of phage selection, the selected scFv antibodies were sublconed into the yeast display vector as described in Example 5. The selected scFv genes were amplified with specific primers that introduced an overlap with the yeast display vector pDNL6. The vector and the fragments were cotransformed into yeast cells to allow cloning by gap repair (Boder and Wittrup, Biotechnol Prog, 14(1):55-62, 1998).

The yeast mini-libraries obtained were further enriched for binders by one or two rounds of sorting using flow cytometry according to published methods (Boder and Wittrup, Biotechnol Prog, 14(1):55-62, 1998). After induction, 2×10⁶ yeast cells were stained with 200 nM of biotinylated antigen. Cells were labeled with streptavidin-AlexaFluor633 to detect binding of biotinylated target antigens and anti-SV5-PE to assess scFv display levels. For the second sort neutravidin-AlexaFluor633 was used to replace conjugated streptavidin and eliminate the chance to sort for streptavidin binders. Yeast clones showing both antigen binding (AlexaFluor633 positives) and display (PE positives) were sorted. The collected cells were grown at 30° C. for 2 days and induced for the next round of sorting at 20° C. for 16 hours. All the described flow cytometry experiments were performed using the FACSAria (Becton Dickinson). After the second sort, single clones were screened for specificity using the specific target and the remaining unrelated antigens and conjugated streptavidin as negative controls.

Affinity measurements were carried on for the best clones according to published methods (Feldhaus et al., Nat Biotechnol, 21(2):163-70, 2003). All the experiments on single clones were performed in a 96-well format using with the LSRII (Becton Dickinson) flow cytometer.

### Production of Polyclonal Antibodies (Yeast and CHO Cells)

After one and two rounds of yeast sorting, the antibody outputs were subcloned into the yeast expression vector pDNL9-RMR (Rabbit Minibody for Recombination) to allow for the expression and secretion of the scFvs as rabbit Fc fusions (minibody) (Di Niro et al., BMC Biotechnol, 7:46, 2007) into the culture supernatant. pDNL6 and pDNL9-RMR were designed to have compatible ends to promote the in vivo homologous recombination of the PCR amplified yeast display library into the yeast expression vector. YVH10 yeast cells were used for the expression in solution of the minibody libraries. Yeast antibody expression was performed following secretion protocols depicted in Wentz and Shusta (Wentz and Shusta, Biotechnol Prog, 24(3):748-56, 2008), using SGT as induction media (50 g tryptone, 1.7 g yeast nitrogen base, 5.3 g ammonium sulfate, 20 g galactose, 1 g dextrose, 10.19 g Na₂HPO₄-7H₂O, 8.56 g NaH₂PO₄—H₂O). Culture supernatant was used directly as a reagent in ELISA and Western Blot.

The pMB-SV5 vector (Di Niro et al., BMC Biotechnol, 7:46, 2007) was modified to allow the production of scFv in fusion with rabbit Fc domain in eukaryotic cells lines. The antibody outputs were PCR amplified and cut with the restriction enzymes BssHII/NheI to allow the cloning into the pMB-SV5 vector. The obtained libraries were transformed in DH5αF′ bacteria cells to produce DNA to transfect into Free-Style™ CHO-S cells (Invitrogen). DNA transfection and minibody production were performed following the manufacturer protocol.

### ELISA Screening of Antibodies

ELISA was performed by coating 96-well ELISA plates (Nunc) with 10 μg/ml neutravidin diluted in 1×PBS, and incubating the plate overnight at 4° C. Wells were blocked with 2% MPBS (1×PBS, 2% skim milk (w/v)) and incubated with 10 μg/ml of biotinylated proteins. After a rinse step, supernatants (obtained by yeast and CHO cells) diluted 1:5 in 1×PBS were added to the wells, followed by HRP-conjugated goat anti-rabbit antibody (Santa Cruz). Immunocomplexes were revealed by adding TMB (Thermo) and reading the plate at 450 nm.

### Immunoblotting on Purified Antigens and Cell Extracts

Biotinylated proteins were separated by SDS-PAGE (Invitrogen) and transferred onto nitrocellulose membranes (GE Healthcare). The membranes were blocked using 2% MPBS for 1 hour at room temperature, then incubated 1 hour with yeast supernatants, diluted 1:1 in 2% MPBS. After extensive washes, the membranes were incubated with AP-conjugated goat anti-rabbit antibody (Santa Cruz), and the immune-complexes revealed by the chromogenic substrate NBT/BCIP (Thermo).

HEK293 cell lysates were also used to test the polyclonal antibodies. 30 μg were used as source of antigens in the immunoblot assay. The detection of immune complexes (specific antigen and the binding activity of the expressed polyclonal antibodies) was obtained with HRP-conjugated goat anti-rabbit antibody (Santa Cruz) and chemiluminescence substrates (Pierce).

### Deep Sequencing: Sample Preparation and Data Analysis

454 GS FLX (Roche) and IonTorrent PGM (Life Technologies) were used for deep sequencing of YAPs. In both cases, barcoded amplicon libraries were prepared using plasmid DNA of the selection outputs as templates. Two different sets of primers were used for each platform. For 454 sequencing, barcoded vector-specific primers were designed as reverse primers, while a unique forward primer mapping on the scFv linker was used. For Ion Torrent, a set of 18 forward primers mapping upstream of CDR3 region was used in combination a vector specific reverse primer containing specific barcodes for each selection output. The amplicons were processed according to the Titanium and Ion 316 protocols.

The raw sequences (sff) of 454 and Ion Torrent were loaded into mothur. The fasta and quality files were extracted and then the sequences were quality trimmed using qwindowsize=50 nd qwindowaverage=22, minlength=50 and maxhomop=8 quality settings. The selection output sequences were parsed using the primer barcodes. The HCDR3 sequences were extracted, the HCDR3DNA sequences were translated into amino acid sequences and imported into a MySQL database table. The unique HCDR3 sequences were clustered using this mysql command: INSERT into bins_table SELECT seq, Count(*) FROM seq_table GROUP BY seq. The unique HCDR3s were clustered by a full outer join of the unique HCDR3s tables. The cluster tables were exported as csv files and further analyzed in Microsoft Excel.

### Results

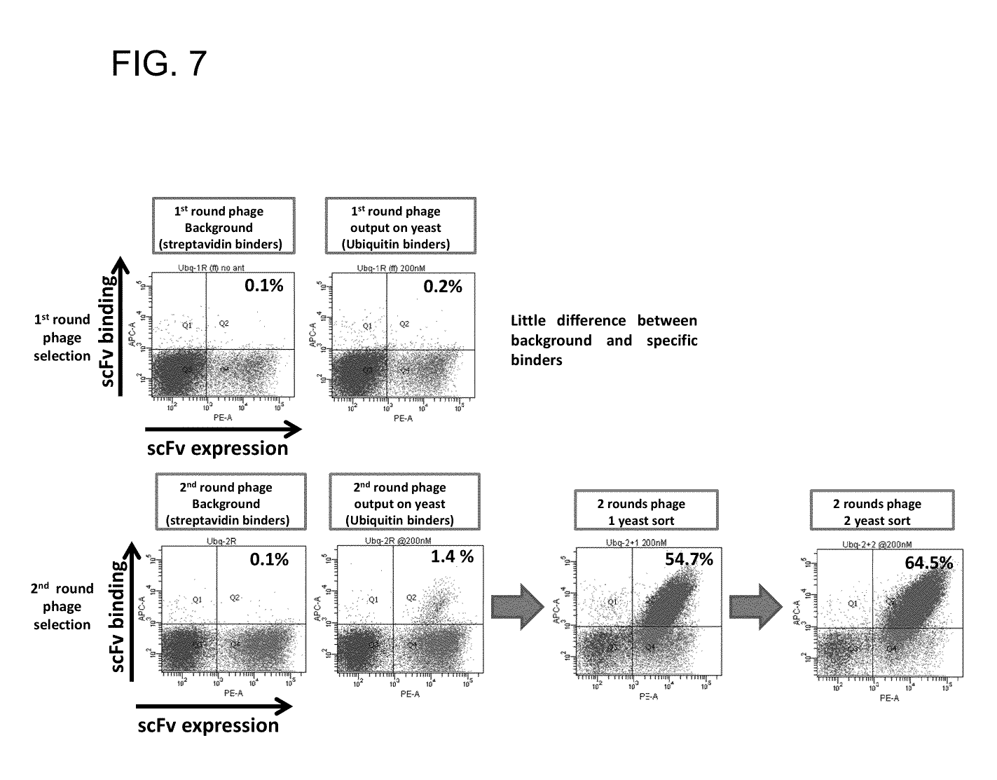

As demonstrated in Example 5, combining phage and yeast display is a powerful method to significantly increase the number of specific antibodies selected against targets, with essentially all antibodies recognizing the target after two rounds of phage selection and two rounds of yeast sorting. Based on this observation, it was hypothesized that the antibody mixtures obtained after such selections could be extremely effective as recombinant renewable polyclonal antibodies (rrpAbs). This hypothesis was tested BY carrying out one or two rounds of phage selection against ubiquitin, and cloning the output into a yeast display vector. As shown in FIG. 7, when a single round of phage selection was carried out, and the antibodies displayed on yeast, only 0.2% of clones recognized ubiquitin (found in Q2), compared to 0.1% found in Q2 in the absence of antigen. This agrees with previous estimates of positive clone abundance after a single phage selection round (de Wildt et al., Nat. Biotechnol. 18, 989, 2000).

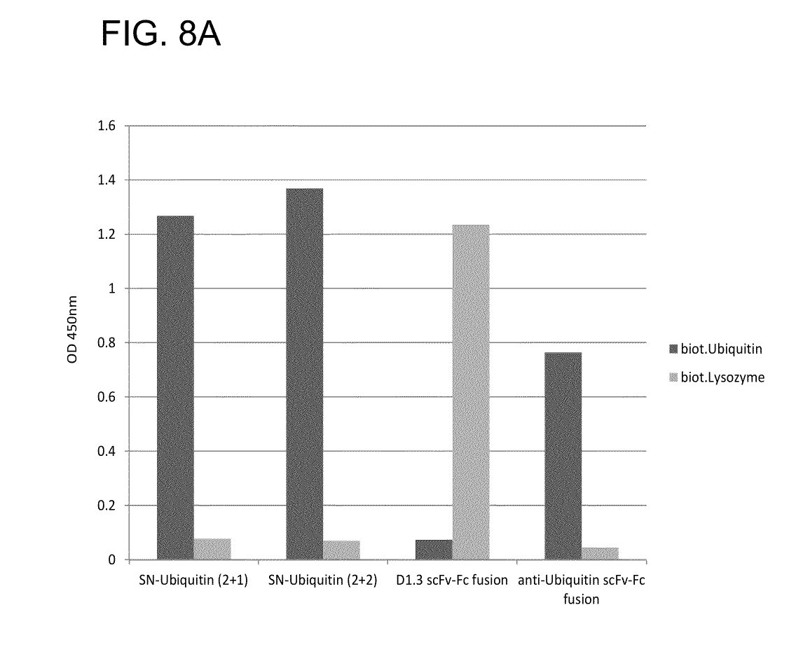

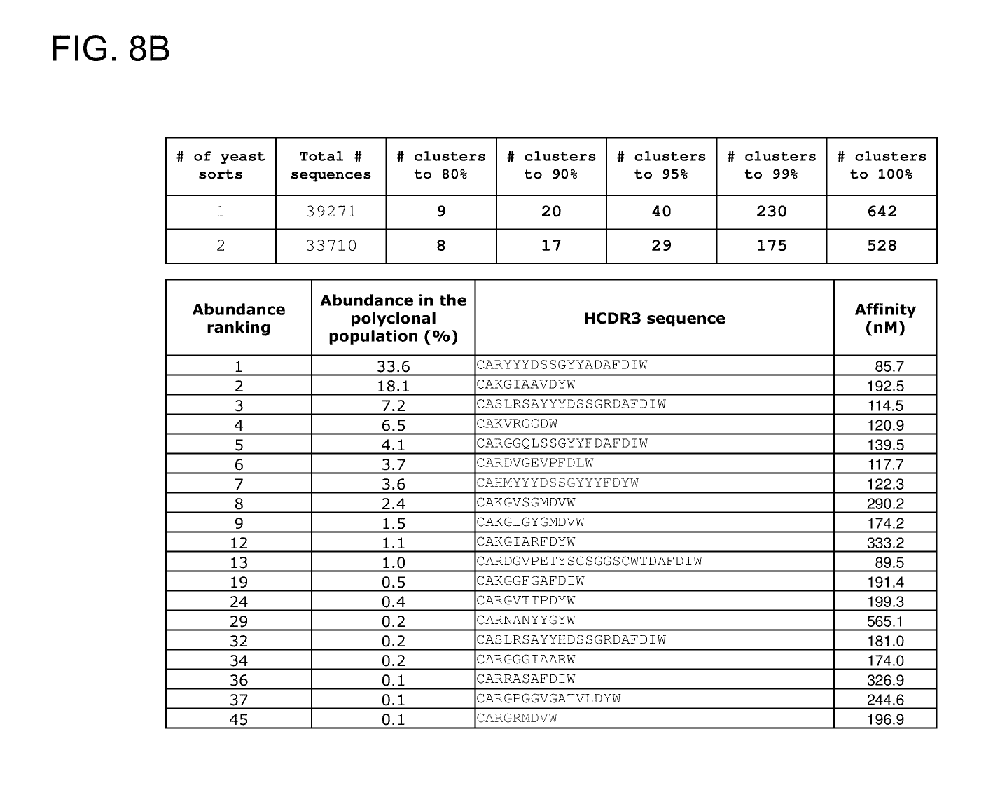

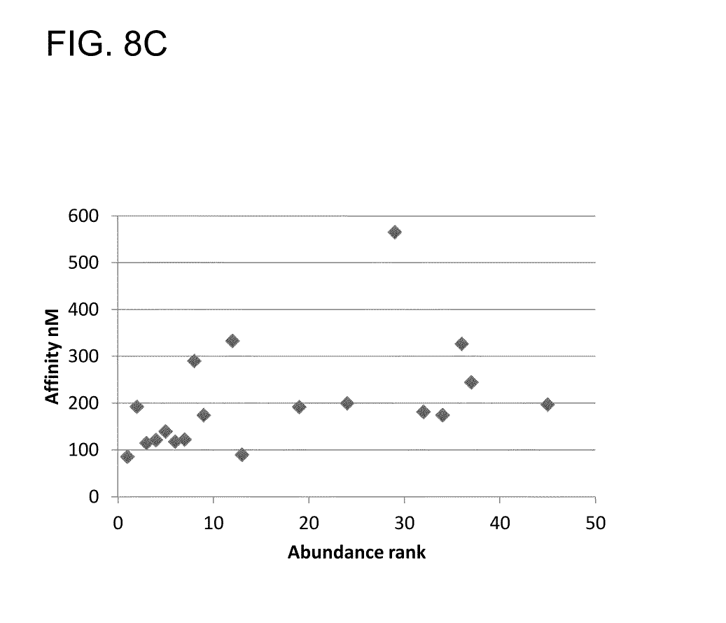

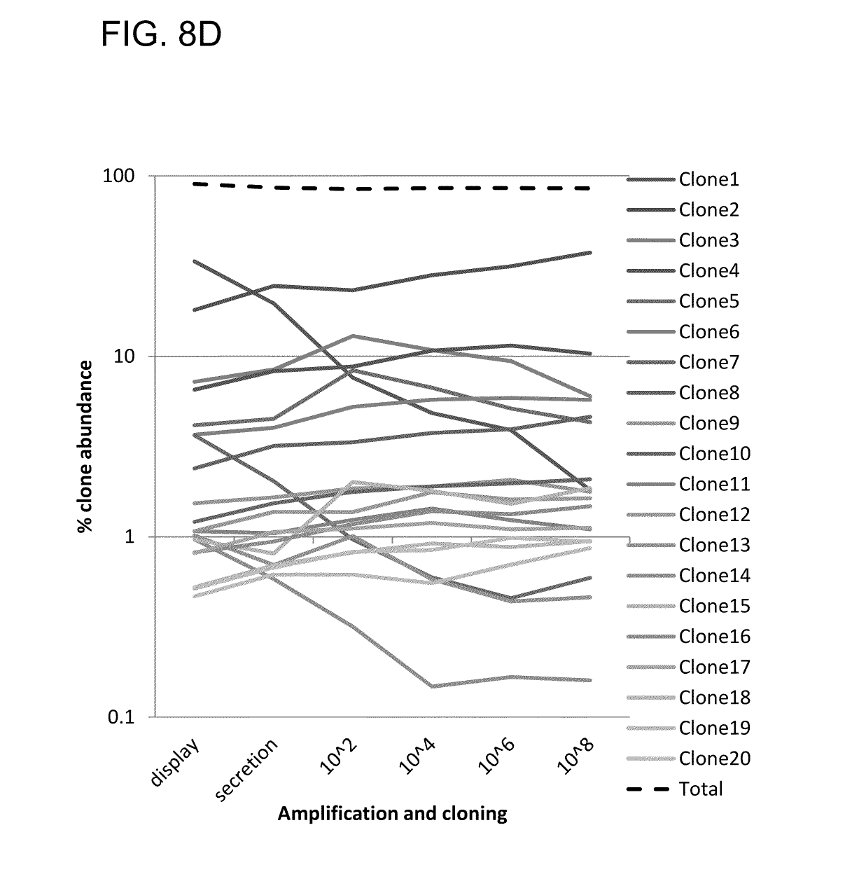

When the output of two rounds of phage selection is displayed on yeast, 1.4% of clones are positive, with the same background of 0.1%. Given these distributions, two rounds of phage selection was chosen as the yeast display input, as this could be sorted far more rapidly. After one round of yeast sorting, the percentage of positive clones increased to 54.7%, rising a further 10% to 64.5% after an additional sorting round. Both of these sorting outputs were subcloned into a yeast secretion vector as polyclonals, in which the scFv was fused to a rabbit Fc domain, and assessed for antigen recognition and specificity. As shown in FIG. 8A, they were similar in their recognition patterns, with two rounds showing slightly higher specific signals. The diversity of the two populations was assessed, by Ion Torrent sequencing, using an algorithm that identifies HCDR3s. 30-40,000 reads were obtained for each polyclonal, and the number of different HCDR3 clusters for each comprised a total of 643 for a single round of yeast sorting, and 528 for two rounds. 95% and 99% of these sequences were accounted for by 40/29 and 230/175 clusters respectively (FIG. 8B). Nineteen yeast clones, including the nine most abundant, were isolated from both sorting rounds (FIG. 8B), and the affinities of their displayed antibodies determined by flow cytometry (Boder et al., Proc Natl Acad Sci USA 97, 10701, 2000).

The affinities of the antibodies comprising the polyclonal population range from 85.7 to 565 nM, with a mean of 203 nM, reflecting the antigen concentration used for selection and sorting (100 nM). All of the isolated antibodies, including the least abundant tested clone (ranked at 45) with an abundance of 0.1%, recognized ubiquitin and not the negative control. The most abundant clone had the highest affinity (85.7 nM), but apart from this antibody the correlation between abundance ranking and affinity was relatively weak (FIG. 8C). These data show that the polyclonal population, as well as the monoclonals that comprise it, are highly specific for ubiquitin.

The yeast producing the rrpAbs were next amplified four times (100-fold each time), for a total 10⁸ fold amplification. After the cloning from the yeast display to the yeast secretion vector, and after each amplification, the antibodies were sequenced by Ion Torrent and the HCDR3s clustered. As shown in FIG. 8D, the top 20 ranked antibodies maintain their approximate percentage abundance over the full one hundred million fold amplification. 80% of the top 20 antibodies show a less than two fold variation after 10⁸ amplification, 15% show less than a four-fold variation, and the worst antibody was reduced by only ten-fold, representing a growth disadvantage of ˜8% for each division. This demonstrates that for all practical purposes, this truly represents a renewable recombinant polyclonal antibody that can be repeatedly used for research purposes.

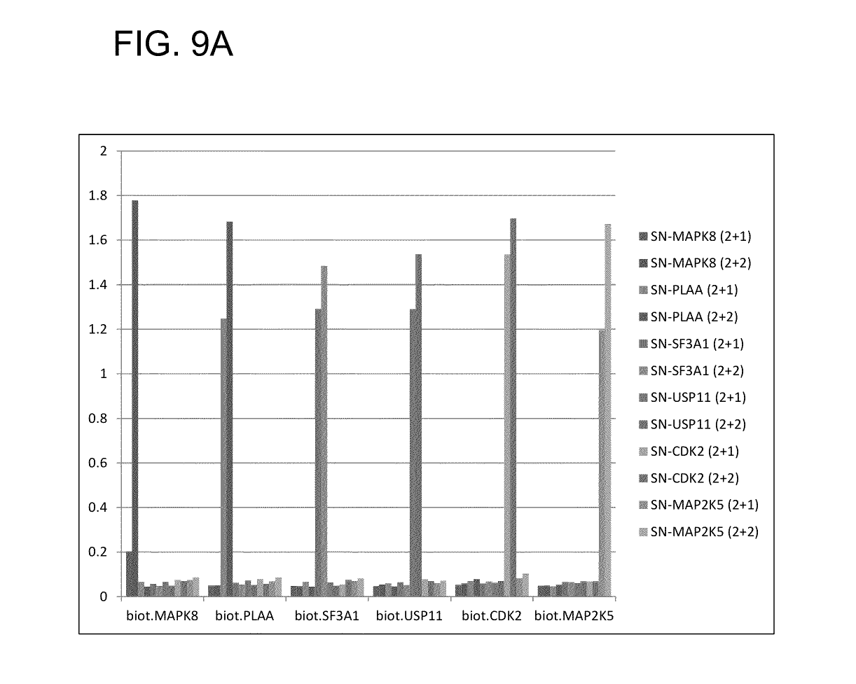

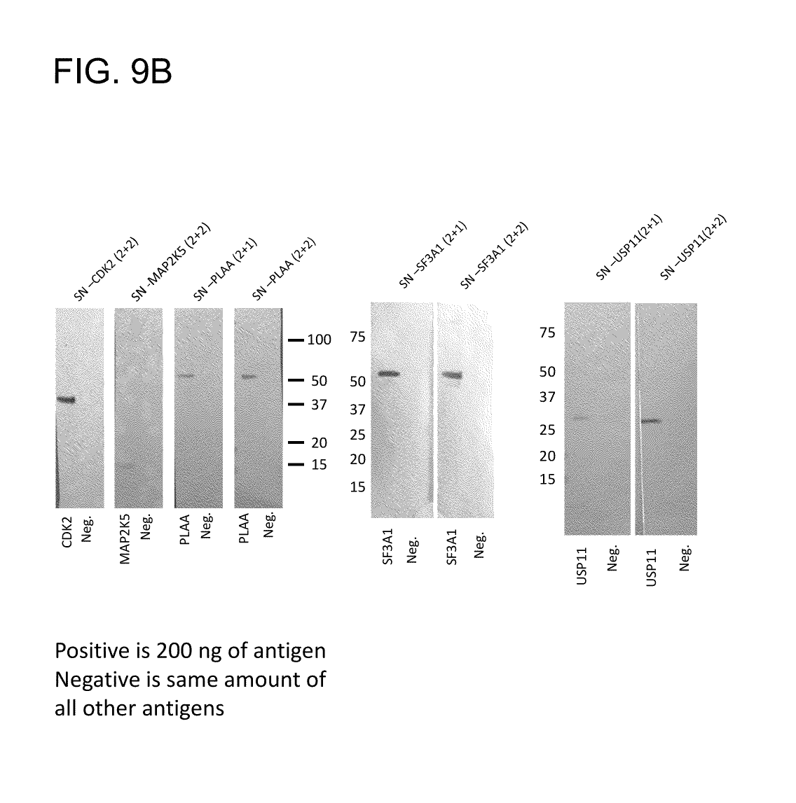

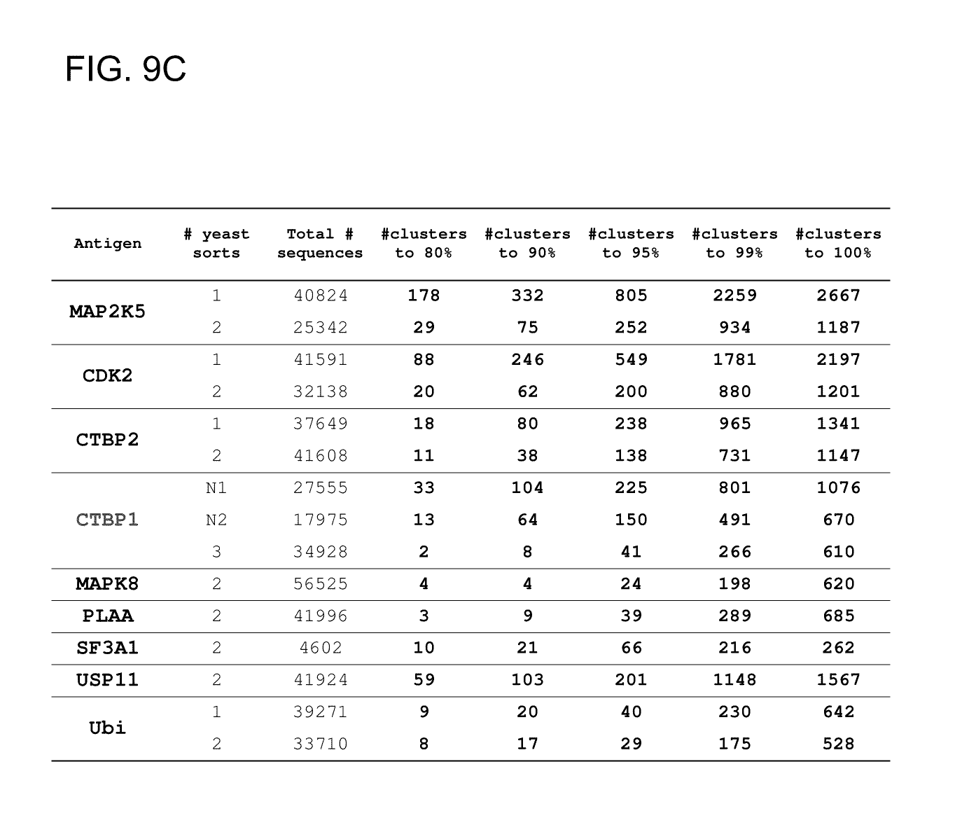

Next, antibodies against eight different human targets were selected using two rounds of phage and one or two rounds of yeast sorting. Outputs were sequenced after each sorting round and recloned for secretion in the scFv-Fc format. Binding specificity was tested by yeast display and ELISA, as shown in FIG. 9A. All rrpAbs recognized their targets specifically in both formats, with excellent signal to noise ratios. In general, the signals with two rounds of yeast sorting were slightly better than with a single round, reflecting the higher percentage of positive yeast cells. However, this increase in positivity was correlated with a reduction in diversity (FIG. 9B): 99% of all antibody sequences were comprised by 230-2259 different HCDR3 clusters after one round of yeast sorting and 175-1148 clusters after two rounds.

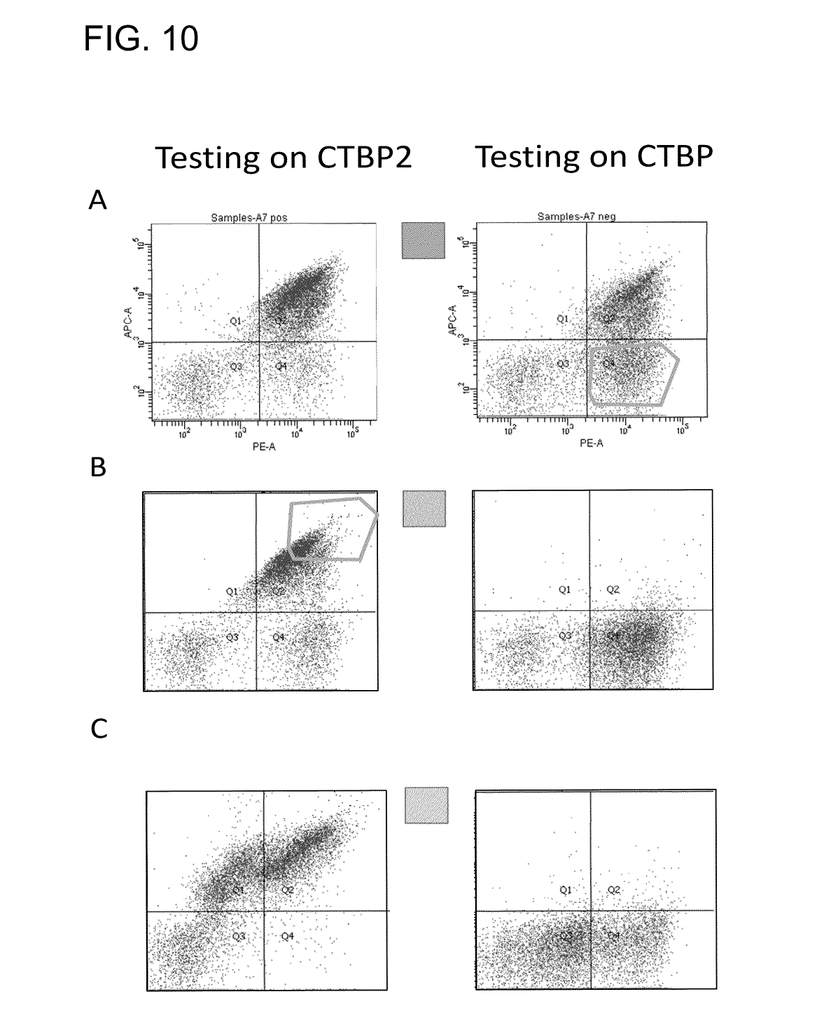

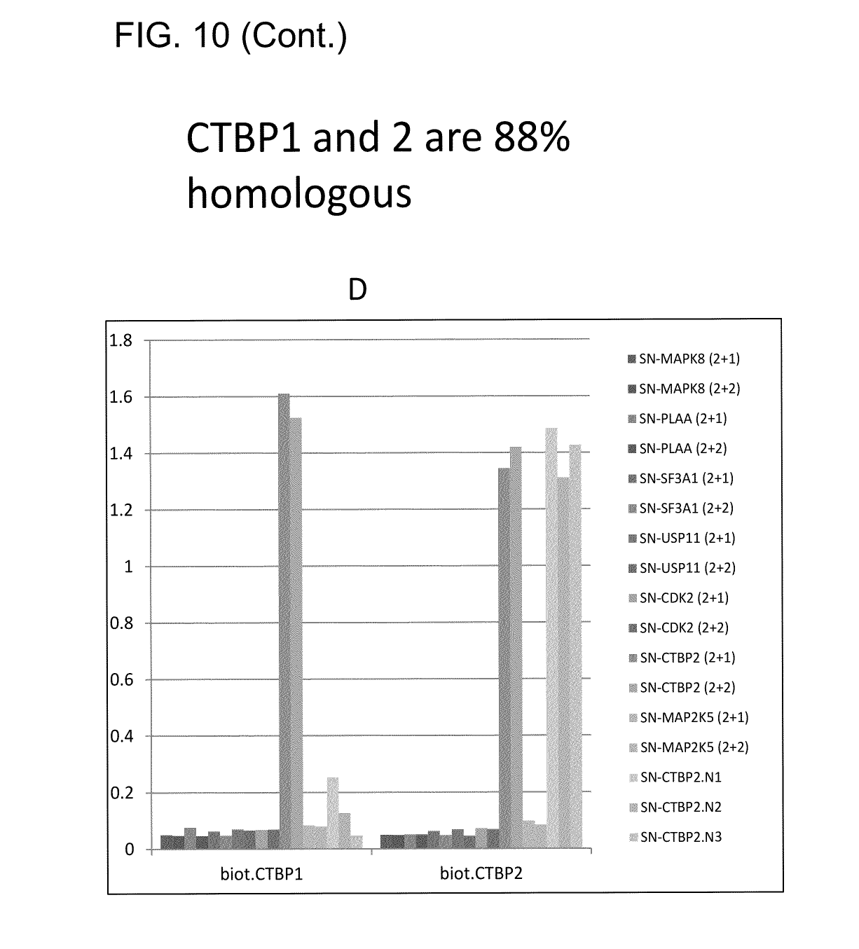

Many proteins encoded in the human genome are duplicated, making it difficult to generate truly specific antibodies. CTBP2 for example, has 88% homology to CTBP1, and a first polyclonal selected against CTBP2 also recognized CTBP1 in flow cytometry (FIG. 10A). However, after two rounds of sorting to eliminate yeast binding to CTBP1 (FIG. 10B), and an additional round sorting for those binding to CTBP2 (FIG. 10C), the activity against CTBP1 was completely eliminated, while that against CTBP2 was maintained (FIGS. 10C and 10D). As expected, the antibody sequences comprising the CTBP2 specific rrpAb showed reduced diversity, compared to that recognizing CTBP1 as well (FIG. 9C), with the number of clusters comprising 99% of antibodies reduced from 731 (CTBP2 2) to 266 (CTBP1 3).

Finally, the functionality of these rrpAbs was assessed. As expected, this was considerably facilitated by the use of the rabbit Fc domain in the scFv-Fc fusions.

### Discussion

An ideal research antibody would be highly specific for its target, without any cross-reactivity or non-specific binding. It would be very sensitive, usable in all assays and would be infinitely renewable. Furthermore, should any properties be less than ideal, it would be possible to improve them relatively easily. Finally, it would be possible to generate such antibodies on a proteomic scale relatively easily.

The development of renewable recombinant polyclonal antibodies (rrpAbs) described herein fulfills all of these criteria. High specificity is a result of the fact that essentially all antibodies recognize the target. This mirrors the findings of Nilsen et al. that affinity purified polyclonal antibodies, in which the vast majority of antibodies recognize the target of interest, give far less background than traditional unfractionated polyclonals in which only 0.5-5% of the antibodies recognize the target (Nilsen et al., Proteomics 5, 4327, 2005). By virtue of their polyclonal nature, these antibodies function at least in ELISA, western blotting and flow cytometry. The ability to completely inhibit the Gall promoter and hence antibody expression during yeast display and secretion, allows the enormous amplification demonstrated here. The availability of the recombinant polyclonal pool within the context of a yeast display vector, makes it very straightforward, as demonstrated with the CTBP1/2 example, to modify the polyclonal properties to eliminate undesired specificities, such as those antibodies recognizing CTBP1 in the CTBP2 population. However, unlike natural polyclonals, in which each positive or negative selection reduces the amount of available antibody, when the recombinant approach is used, desired antibodies are easily reamplified after each selection, so that the total amount of antibody remains high and the proportion of antibodies redirected towards the new specificity is increased.

scFv-Fc fusions were created using the rabbit Fc. These recombinant molecules are recognized by protein A, as well as traditional rabbit secondary reagents, allowing their direct substitution for traditional rabbit polyclonals. As they are predominantly directed towards the target of interest (and have been characterized) it is expected that they will be better than most commercially available polyclonals. Furthermore, although rabbit Fc domains have been used herein, it would be trivial to substitute other Fc domains of interest.

An additional significant advantage to this approach is that the whole selection process for a single recombinant polyclonal antibody against a target can be retained within the single well of a microtiter plate: there is no need to deconvolute one well to a full microtiter plate to select specific monoclonals. This should significantly reduce the cost of antibody selection, and render feasible the goal of selecting renewable antibodies against all human proteins (Taussig et al., Nature Methods 4, 13, 2007). While most research applications will be able to use rrpAbs, they also serve as a potent source of monoclonal antibodies should they be required. As shown in Example 5, it is relatively straightforward to screen large numbers of monoclonals for desired properties, since essentially all antibodies making up the rrpAbs are specific for the target.

The combination of phage and yeast display described herein harnesses the advantages of both platforms. The initial phage antibody library has a diversity of >10¹⁰ (Sblattero and Bradbury, Nat. Biotechnol. 18:75, 2000). By carrying out two rounds of phage display, it was possible to capture those antibodies showing some reactivity for the target. This reduces the diversity to 10⁵⁻⁶, which is compatible with yeast display cloning, and allows further sorting to restrict diversity to those clones recognizing the target of interest. When one round of yeast sorting was carried out, 99% of the antibody sequences were comprised by 175 to 2259 different HCDR3 clusters. True diversity, which will also encompass different VL genes, and additional VH differences, is likely to be significantly higher. After carrying out an additional round of sorting, or eliminating the CTBP1 binding antibodies, the diversity still remains very high with 266 different HCDR3 clusters identified. It is the great initial depth of these polyclonal pools that permits the reamplification of subsets with specific recognition properties. In fact, this initial diversity probably exceeds that occurring during an immune response, which for tetanus toxoid has been estimated to be 100 different VH/VL clone combinations (Poulsen et al., J Immunol 179, 3841, 2007).

In summary, the approach described herein makes possible the selection of highly specific antibodies against all proteins encoded by the human genome.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of generating a renewable polyclonal antibody population specific for a target antigen, comprising:
providing a collection of antibody clones;
selecting from the collection a population of antibody clones that bind the target antigen;
transferring the selected population of antibody clones into a yeast display vector to generate a yeast display antibody library; and
selecting from the yeast display antibody library a population of yeast clones that specifically bind the target antigen to generate a target-specific polyclonal antibody population, thereby generating a renewable polyclonal antibody population specific for the target antigen.

2. The method of claim 1, further comprising expressing the target-specific polyclonal antibody population in an expression vector.

3. The method of claim 2, wherein the antibodies are expressed as scFvs, Fabs, scFv-Fc fusion proteins or full length immunoglobulins.

4. The method of claim 3, wherein the antibodies are expressed in yeast, Escherichia coli, or mammalian cells.

5. The method of claim 1, further comprising amplifying the target-specific polyclonal antibody population, wherein amplification of the target-specific polyclonal antibody population maintains at least 70% of the original diversity of the individual yeast clones.

6. The method of claim 5, wherein the polyclonal antibody population is amplified at least 10²-fold, 10³-fold, 10⁴-fold, 10⁵-fold, 10⁶-fold, 10⁷-fold or 10⁸-fold and maintains at least 70% of the original diversity of the individual yeast clones.

7. The method of claim 6, wherein at least 75%, at least 80% or at least 85% of the original diversity of the individual yeast clones is maintained following amplification.

8. The method of claim 1, wherein the polyclonal antibody population comprises at least 100, at least 150, at least 200, at least 250 or at least 300 different antibodies.

9. The method of claim 1, wherein the collection of antibody clones comprises an antibody display library.

10. The method of claim 9, wherein the antibody display library comprises a naïve antibody library.

11. The method of claim 9, wherein the antibody display library comprises a phage display library, a ribosome display library, a bacterial display library or an in vitro display library.

12. The method of claim 9, wherein the antibody display library is a single domain VH, VHH, VL, scFv or Fab library.

13. The method of claim 12, wherein the antibody display library is a natural library.

14. The method of claim 12, wherein the antibody display library is a synthetic antibody library.

15. The method of claim 1, wherein selecting a population of antibody clones from the collection comprises at least one, at least two, at least three, at least four or at least five rounds of selection against the target antigen.

16. The method of claim 15, wherein selecting a population of antibody clones from the collection comprises no more than two rounds of selection against the target antigen.

17. The method of claim 1, wherein the collection of antibody clones comprises a phage display library and selecting the population of antibody clones from the collection comprises no more than two rounds of selection against the target antigen using phage display.

18. The method of claim 1, wherein selecting a population of yeast clones from the yeast display antibody library comprises at least one, at least two, at least three, at least four or at least five rounds of selection against the target antigen.

19. The method of claim 18, wherein selecting a population of yeast clones from the yeast display antibody library comprises no more than two rounds of selection against the target antigen.

20. A polyclonal antibody or a polyclonal antibody population produced by the method of claim 1.

21. A method of generating a renewable polyclonal antibody population specific for a target antigen, comprising:
providing a collection of antibody clones in a first display platform;
selecting from the collection a population of antibody clones that bind the target antigen to generate a first antibody display library;
transferring the selected population of antibody clones into a second display platform to generate a second antibody display library; and
selecting from the second antibody display library a population of clones that specifically bind the target antigen to generate a target-specific polyclonal antibody population, thereby generating a renewable polyclonal antibody population specific for the target antigen.

22. The method of claim 21, wherein the first display platform and the second display platform are selected from phage display, yeast display, ribosome display, bacterial display and in vitro display.
